(19) United States
(12) Patent Application Publication
(10) Pub. No.: US 2026/0151262 A1
(43) Pub. Date: Jun. 4, 2026

(54) ENHANCED SEPTUM RETENTION FOR IMPLANTED OCULAR DELIVERY SYSTEMS

(71) Applicant: Genentech, Inc., South San Francisco, CA (US)

(72) Inventors: Joshua Horvath, South San Francisco, CA (US); Brooke Lohman, South San Francisco, CA (US)

(21) Appl. No.: 19/459,352

(22) Filed: Jan. 26, 2026

Related U.S. Application Data
(63) Continuation of application No. PCT/US2024/039557, filed on Jul. 25, 2024.
(60) Provisional application No. 63/516,450, filed on Jul. 28, 2023.
(60) Provisional application No. 63/595,615, filed on Nov. 2, 2023.

(51) Int. Cl. A61F 9/00

(52) U.S. Cl. CPC A61F 9/0017

(57) ABSTRACT

An ophthalmic drug delivery device including a body defining a refillable reservoir; an extrascleral flange projecting from a proximal end of the body and defining a bore extending from an upper surface of the flange into the reservoir; a septum including an upper surface connected to a lower surface by a curved outer surface. The curved outer surface of the septum forms a first bond with the bore. A perimeter region of the upper surface of the septum lies flush with a plane of the upper surface of the flange and a central region of the upper surface lies below the perimeter region. An elastomeric encasement extends over at least the upper surface of the flange and the upper surface of the septum and prevents displacement of the septum relative to the bore upon penetration by a needle. Related devices, systems, and methods are provided.

### CROSS-REFERENCE TO PRIORITY DOCUMENTS

This application is a bypass continuation of International Patent Application No. PCT/US 2024/039557, filed Jul. 25, 2024, which claims priority to U.S. Provisional Patent Application No. 63/516,450, filed Jul. 28, 2023; and to U.S. Provisional Patent Application No. 63/595,615, filed Nov. 2, 2023. The disclosures of each of the above-noted applications is incorporated by reference, in its entirety.

### BACKGROUND

Diseases that affect vision can be treated with a variety of therapeutic agents, but the delivery of drugs to the eye continues to be challenging.

Injections of therapeutic via the eye can be painful, involve some risk of infection, hemorrhage and retinal detachment. Depending on the frequency, intra-ocular injections can be time-consuming for both patient and physician. Consequently, in at least some instances the drug may be administered less often than the prescribed frequency resulting in sub-optimal treatment benefit. Further, bolus intra-ocular injections may not provide the ideal pharmacokinetics and pharmacodynamics. A bolus injection of drug into the vitreous humor of a patient can result in a peak drug concentration several times higher than the desired therapeutic amount and then before the patient is able to get the next injection drop to a drug concentration that is far below therapeutic effectiveness.

Implant devices provide sustained release of a therapeutic drug permitting long-term therapy with fewer injections of the eye. Some implant devices are capable of being refilled while at least partially implanted in the eye allowing for even longer therapy with the same implant device. A needle device can be used to refill the implanted device by penetrating a region of the implanted device. Repeated penetration of the implanted device using a needle device can be problematic for device retention and structural integrity necessary for long-term therapy.

### SUMMARY

In an aspect, described is an ophthalmic drug delivery device including a body defining a refillable reservoir. The device includes an extrascleral flange projecting from a proximal end of the body and defining a bore extending from an upper surface of the flange into the reservoir defined by the body and a septum having an upper surface connected to a lower surface by a curved outer surface. The curved outer surface of the septum forms a first bond with the bore. A perimeter region of the upper surface of the septum lies flush with a plane of the upper surface of the flange and a central region of the upper surface lies below the perimeter region. The device includes an elastomeric encasement extending over at least the upper surface of the flange and the upper surface of the septum. The encasement prevents displacement of the septum relative to the bore upon penetration by a needle.

The septum can be pre-molded to a first shape and cut to a second shape once the septum is positioned within the bore. The second shape can include an exposed interior of the septum, the exposed interior spanning across the full upper surface of the septum. A maximum height of the septum between the lower surface and the upper surface can be greater than 1.05 mm and can be less than 1.35 mm. The central region of the upper surface of the septum can form a depression in the upper surface of the septum, wherein the encasement fills the depression. The encasement and the upper surface of the septum can interface along a full diameter of the septum. A contact area at the interface can be non-planar.

The septum can be formed of silicone elastomer. The body can be formed of polysulfone. The encasement can be formed of silicone elastomer. The encasement can bond only to the upper surface of the septum forming a second bond. The first bond between the curved outer surface of the septum and the bore can be formed using an epoxy adhesive. The epoxy adhesive can include an amine-based epoxy curing agent. The first bond can be substantially free of trimethyl-1,6-Hexandiamine prior to forming the second bond between the encasement and the upper surface of the septum.

The first bond between the curved outer surface of the septum and the bore can be cured in at least two stages prior to forming the second bond between the encasement and the upper surface of the septum. A first stage of curing of the at least two stages can include heating the first bond to about 67° C. for at least 1 hour up to about 3 hours. A second stage of the at least two stages of curing can include heating the first bond to at least 115-135° C. for more than 30 minutes and less than about 4 hours.

The body can be transparent or translucent. The encasement can be transparent or translucent. The encasement and the septum can be configured to be penetrated by the needle during refilling of the reservoir and configured to reseal after penetration and upon removal of the needle from the reservoir. The needle can be siliconized along at least a portion of its length. The septum can be oversized relative to the bore such that the bore applies radial compression on the septum. The radial compression can encourage re-sealing of the septum after penetration and upon removal of the needle from the septum. The body defining the refillable reservoir can be configured for implantation within a vitreous of an eye through a penetration in the sclera of the eye. The body can further include a porous structure positioned within a distal end region of the refillable reservoir. The reservoir can have a volume sized to contain an amount of a therapeutic formulation. The porous structure can be configured to control diffusion of the therapeutic from the reservoir into the eye. The encasement can encapsulate the upper surface and a lower surface of the flange without bonding to the flange. The flange can further include one or more through-holes.

In some variations, one or more of the following can optionally be included in any feasible combination in the above methods, apparatus, devices, and systems. More details are set forth in the accompanying drawings and the description below. Other features and advantages will be apparent from the description and the drawings.

### BRIEF DESCRIPTION OF THE DRAWINGS

These and other aspects will now be described in detail with reference to the following drawings. Generally speaking, the figures are not to scale in absolute terms or comparatively but are intended to be illustrative. Also, relative placement of features and elements may be modified for the purpose of illustrative clarity.

FIG. 1 is a cross-sectional, schematic view of a portion of the human eye;

FIG. 2 is a cross-sectional, schematic view of a portion of the human eye having an implementation of a therapeutic device implanted therein;

FIG. 3A is a side view of an implementation of a therapeutic device;

FIG. 3B is a cross-sectional view of the therapeutic device of FIG. 3A taken along line B-B;

FIG. 3C is a side view of the device of FIG. 3A rotated 90 degrees;

FIG. 4A is a cross-sectional view of a proximal end region of a device illustrating a septum having an upper surface including an untrimmed outer perimeter region;

FIG. 4B is a CT image of a proximal end region of a device having a septum having an upper surface including an untrimmed outer perimeter region in which separation between the untrimmed outer perimeter region of the septum and the encasement occurred due to poor bonding;

FIG. 4C is a CT image of a proximal end region of a device illustrating a septum having an upper surface including an untrimmed outer perimeter region as in FIG. 4A;

FIG. 5A is a cross-sectional view of a proximal end region of the device of FIG. 3B illustrating the fully trimmed upper surface of the septum;

FIG. 5B is a CT image of a proximal end region of a device having a fully trimmed upper surface of the septum bonded to the encasement;

FIG. 5C is a CT image of a proximal end region of a device without an encasement illustrating the fully trimmed upper surface of the septum as in FIG. 5A;

FIG. 6A is a side view of an exchange needle apparatus;

FIG. 6B is a detail view of an elongate structure of the refill needle and hub of FIG. 6A;

FIG. 6C is a cross-sectional view of the elongate structure of FIG. 6B;

FIG. 6D is a cross-sectional view of the connector of the refill needle to couple to a syringe;

FIG. 6E is a detail view of an elongate needle of the exchange needle apparatus having a lubricating coating to reduce the force for septum penetration;

FIG. 7A shows GCMS data showing significantly higher Trimethyl-1,6-Hexanediamine (TMHMD) residuals within the proximal bodies in which no second epoxy cure was performed;

FIG. 7B shows GCMS data showing significantly lower signals below limit of detection (LOD) for TMHMD residuals within the proximal bodies of a sample in which a second epoxy cure at 150° C. for 3 hours was performed;

FIG. 7C shows GCMS data showing significantly lower signals below LOD for TMHMD residuals within the proximal bodies of a sample in which a second epoxy cure at 125° C. for 3 hours was performed;

FIG. 8A shows overmold separation force in Newtons (N) for an implant without a second epoxy cure compared to an implant having a second epoxy cure at 115-135° C. for 3 hours and an implant having a second epoxy cure at 115-135° C. for 0.5 hours illustrating the second epoxy cure significantly increases the force necessary to separate the overmold from the septum depending on the length of the curing step;

FIG. 8B shows overmold separation force in Newtons (N) for implants with a second epoxy cure at 115° C. and 135° C. for 0.5 hours compared to 3 hours illustrating the effect of time over temperature on the separation force;

FIG. 9A shows performance of implants repeatedly penetrated by an uncoated refill needle;

FIG. 9B shows reducing penetration force by siliconizing a refill needle improves septum retention and implant survival upon repeated penetration by the refill needle;

FIG. 9C shows reducing penetration force by siliconizing a refill needle and increasing the septum height and improving geometry of the upper surface relative to the septum improves septum retention and implant survival upon repeated penetration by the refill needle;

FIG. 9D shows reducing penetration force by siliconizing a refill needle without changing the septum height or geometry improves septum retention and implant survival upon repeated penetration by the refill needle;

FIG. 9E shows the combined effect of reducing penetration force by siliconizing a refill needle, increasing the septum height, improving geometry of the upper surface relative to the septum, reducing epoxy and increasing epoxy curing time improves septum retention and implant survival upon repeated penetration by the refill needle;

FIG. 10 shows a correlation between bond strength of overmolded encasement and septum with punctures-to-failure.

### DETAILED DESCRIPTION

Described herein are implantable devices, systems and methods of use for the delivery of one or more therapeutics for the treatment of diseases.

The implantable therapeutic devices described herein are configured to be repeatedly refilled with therapeutic formulations while positioned within a patient and without explanation of the device. The devices are capable of long-term, minimally-invasive delivery of treatments to the eye. The devices described herein incorporate a septum system that, despite repeated needle penetrations of the septum with a needle, has robust retention relative to the device body. The upper end region of the septum provides a non-planar interface along its entire upper surface that increases the contact area between the septum and an overmold or elastomeric cover or encasement encapsulating the proximal end region of the device. The septum geometry avoids gaps between its perimeter region and the access opening of the device body thereby improving the bond between the septum and the outer encasement. The bond between the septum and the access opening of the device is additionally improved by removing all residual Trimethyl-1,6-hexanediamine (TMHMD) from the septum so that the first bond is substantially free of TMHMD prior to forming a second bond between the encasement and the upper surface of the septum. These and other features of the devices will be described in more detail below.

It should be appreciated that the devices and systems described herein can incorporate any of a variety of features described herein and that elements or features of one implementation of a device and system described herein can be incorporated alternatively or in combination with elements or features of another implementation of a device and system described herein as well as the various implants and features described in U.S. Pat. Nos. 8,399,006; 8,623,395; 9,033,911; 10,500,091; and 9,526,654, the entire disclosures of which are incorporated herein by reference thereto. For the sake of brevity, explicit descriptions of each of those combinations may be omitted although the various combinations are to be considered herein. Additionally, described herein are different methods for implantation and access of the devices.

The various implants can be implanted, filled, refilled etc., according to a variety of different methods and using a variety of different devices and systems. Provided are some representative descriptions of how the various devices may be implanted and accessed, however, for the sake of brevity explicit descriptions of each method with respect to each implant or system may be omitted.

It should also be appreciated that the devices and systems described herein can be positioned in many locations of the eye and need not be implanted specifically as shown in the figures or as described herein. The devices and systems described herein can be used to deliver therapeutic agent(s) for an extended period of time to one or more of the following tissues: intraocular, intravascular, intraarticular, intrathecal, pericardial, intraluminal and intraperitoneal. Although specific reference is made below to the delivery of treatments to the eye, it also should be appreciated that medical conditions besides ocular conditions can be treated with the devices and systems described herein. For example, the devices and systems can deliver treatments for inflammation, infection, and cancerous growths. Any number of drug combinations can be delivered using any of the devices and systems described herein.

The materials, compounds, compositions, articles, and methods described herein may be understood more readily by reference to the following detailed description of specific aspects of the disclosed subject matter and the Examples included therein. Before the present materials, compounds, compositions, articles, devices, and methods are disclosed and described, it is to be understood that the aspects described below are not limited to specific methods or specific reagents, as such may vary. It is also to be understood that the terminology used herein is for the purpose of describing particular aspects only and is not intended to be limiting.

### Definitions

Unless defined otherwise, all technical and scientific terms used herein have the same meaning as is commonly understood by one of skill in the art to which the invention(s) belong. All patents, patent applications, published applications and publications, websites and other published materials referred to throughout the entire disclosure herein, unless noted otherwise, are incorporated by reference in their entirety. In the event that there are pluralities of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, relative directional terms such as anterior, posterior, proximal, distal, lateral, medial, sagittal, coronal, transverse, etc. are used throughout this disclosure. Such terminology is for purposes of describing devices and features of the devices and is not intended to be limited. For example, as used herein “proximal” generally means closest to a user implanting a device and farthest from the target location of implantation, while “distal” means farthest from the user implanting a device in a patient and closest to the target location of implantation.

As used herein, a disease or disorder refers to a pathological condition in an organism resulting from, for example, infection or genetic defect, and characterized by identifiable symptoms.

As used herein, treatment means any manner in which the symptoms of a condition, disorder or disease are ameliorated or otherwise beneficially altered. Treatment also encompasses any pharmaceutical use of the devices described and provided herein.

As used herein, amelioration or alleviation of the symptoms of a particular disorder, such as by administration of a particular pharmaceutical composition, refers to any lessening, whether permanent or temporary, lasting or transient that can be attributed to or associated with administration of the composition.

As used herein, an effective amount of a compound for treating a particular disease is an amount that is sufficient to ameliorate, or in some manner reduce the symptoms associated with the disease. Such an amount can be administered as a single dosage or can be administered according to a regimen, whereby it is effective. The amount can cure the disease but, typically, is administered in order to ameliorate the symptoms of the disease. Repeated administration can be required to achieve the desired amelioration of symptoms. Pharmaceutically effective amount, therapeutically effective amount, biologically effective amount and therapeutic amount are used interchangeably herein to refer to an amount of a therapeutic that is sufficient to achieve a desired result, i.e. Therapeutic effect, whether quantitative or qualitative. In particular, a pharmaceutically effective amount, in vivo, is that amount that results in the reduction, delay, or elimination of undesirable effects (such as pathological, clinical, biochemical and the like) in the subject.

As used herein, sustained release encompasses release of effective amounts of an active ingredient of a therapeutic agent for an extended period of time. The sustained release may encompass first order release of the active ingredient, zero order release of the active ingredient, or other kinetics of release such as intermediate to zero order and first order, or combinations thereof. The sustained release may encompass controlled release of the therapeutic agent via passive molecular diffusion driven by a concentration gradient across a porous structure.

As used herein, a subject includes any animal for whom diagnosis, screening, monitoring or treatment is contemplated. Animals include mammals such as primates and domesticated animals. An exemplary primate is human. A patient refers to a subject such as a mammal, primate, human, or livestock subject afflicted with a disease condition or for which a disease condition is to be determined or risk of a disease condition is to be determined.

As used herein, a therapeutic agent referred to with a trade name encompasses one or more of the formulation of the therapeutic agent commercially available under the tradename, the active ingredient of the commercially available formulation, the generic name of the active ingredient, or the molecule comprising the active ingredient. As used herein, therapeutic or therapeutic agents are agents that ameliorate the symptoms of a disease or disorder or ameliorate the disease or disorder. Therapeutic agent, therapeutic compound, therapeutic regimen, or chemotherapeutic include conventional drugs and drug therapies, including vaccines, which are known to those skilled in the art and described elsewhere herein. Therapeutic agents include, but are not limited to, moieties that are capable of controlled, sustained release into the body.

As used herein, a composition refers to any mixture. It can be a solution, a suspension, an emulsion, liquid, powder, a paste, aqueous, non-aqueous or any combination of such ingredients.

As used herein, fluid refers to any composition that can flow. Fluids thus encompass compositions that are in the form of semi-solids, pastes, solutions, aqueous mixtures, gels, lotions, creams and other such compositions.

As used herein, a kit is a packaged combination, optionally, including instructions for use of the combination and/or other reactions and components for such use.

### Eye Anatomy

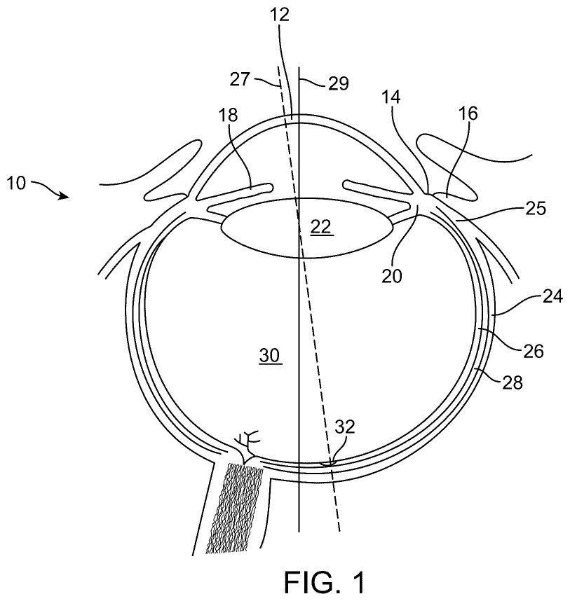

FIG. 1 is a cross-sectional, schematic view of a portion of the human eye 10 showing the anterior chamber, posterior chamber and vitreous of the eye. The eye 10 is generally spherical and is covered on the outside by the sclera 24. The bulk of the eye 10 is filled and supported by the vitreous (referred to herein as vitreous humor or vitreous body) 30, a clear, jelly-like substance disposed between the lens 22 and the retina 26. The retina 26 lines the inside posterior segment of the eye 10 and includes the macula 32. The retina 26 registers the light and sends signals to the brain via the optic nerve. The fovea centralis is the part of the eye located in the center of the macula 32 of the retina 26 and is the region responsible for sharp central vision, for example in order to read or drive. An imaginary line passing from the midpoint of the visual field to the fovea centralis is called the visual axis 27. The hypothetical straight line passing through the centers of curvature of the front and back surfaces of the lens 22 is the optic axis 29.

The elastic lens 22 is located near the front of the eye 10. The lens 22 provides adjustment of focus and is suspended within a capsular bag from the ciliary body 20, which contains the muscles that change the focal length of the lens 22. A volume in front of the lens 22 is divided into two by the iris 18, which controls the aperture of the lens 22 and the amount of light striking the retina 26. The pupil is a hole in the center of the iris 18 through which light entering anteriorly passes. The volume between the iris 18 and the lens 22 is the posterior chamber. The volume between the iris 18 and the cornea 12 is the anterior chamber. Both chambers are filled with a clear liquid known as aqueous humor.

The cornea 12 extends to and connects with the sclera 24 at a location called the limbus 14 of the eye. The conjunctiva 16 of the eye is disposed over the sclera 24 and the Tenon's capsule (not shown) extends between the conjunctiva 16 and the sclera 24. The eye 10 also includes a vascular tissue layer called the choroid 28 that is disposed between a portion of the sclera 24 and the retina 26. The ciliary body 20 is continuous with the base of the iris 18 and is divided anatomically into pars plica and pars plana 25, a posterior flat area approximately 4 mm long.

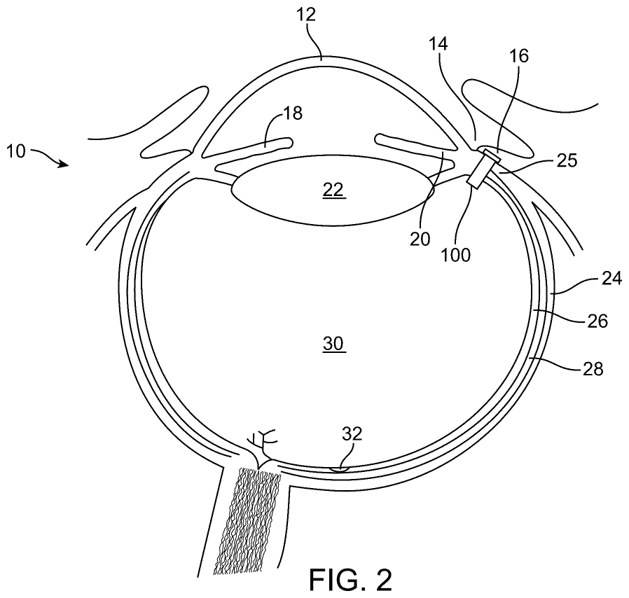

The devices described herein can be positioned in many locations of the eye 10, for example in the pars plana region away from tendon of the superior rectus muscle and one or more of posterior to the tendon, anterior to the tendon, under the tendon, or with nasal or temporal placement of the therapeutic device. As shown in FIG. 2, the devices described herein can be positioned along an axis of insertion A through the sclera 24 in the pars plana region such that the device avoids interfering with the visual field, and in particular, the visual and optic axes 27, 29. The device 100 can be implanted under the conjunctiva 16.

Surgical placement of trans-scleral ocular implants designed to penetrate the globe such that certain regions of the implant occupy supra-scleral, trans-scleral, sub-scleral, and intravitreal aspects of the ocular anatomy in the pars plana region involves a risk of acute vitreous hemorrhage (VH) following surgery. The devices described herein incorporate one or more features that mitigate the risk of vitreous hemorrhage at the time of surgical implantation and improved healing following surgery.

### Treatment Devices

The devices described herein are referred to as drug delivery devices, treatment devices, therapeutic devices, port delivery systems, and the like. It should be appreciated that these terms are used interchangeably herein and are not intended to be limiting to a particular implementation of device over another. The devices and systems described herein can incorporate any of a variety of features described herein and the elements or features of one implementation of a device and system described herein can be incorporated alternatively or in combination with elements or features of another implementation of a device and system described herein as well as the various implants and features described in U.S. Pat. Nos. 8,399,006; 8,623,395; 9,033,911; 10,500,091; and 9,526,654. For the sake of brevity, explicit descriptions of each of those combinations may be omitted although the various combinations are to be considered herein. Additionally, described herein are different methods for implantation and access of the devices. The various implants can be implanted, filled, refilled etc. according to a variety of different methods and using a variety of different devices and systems. Provided are some representative descriptions of how the various devices may be implanted and accessed, however, for the sake of brevity explicit descriptions of each method with respect to each implant or system may be omitted.

The porous structures (also referred to herein as a drug release mechanism, drug release element, release control element, RCE, or frit) as described herein can be used with a number of various different implantable therapeutic devices including one or more of those devices described U.S. Pat. Nos. 8,399,006; 8,623,395; 9,033,911; 10,500,091; and 9,526,654, the entire disclosures of which are incorporated herein by reference thereto.

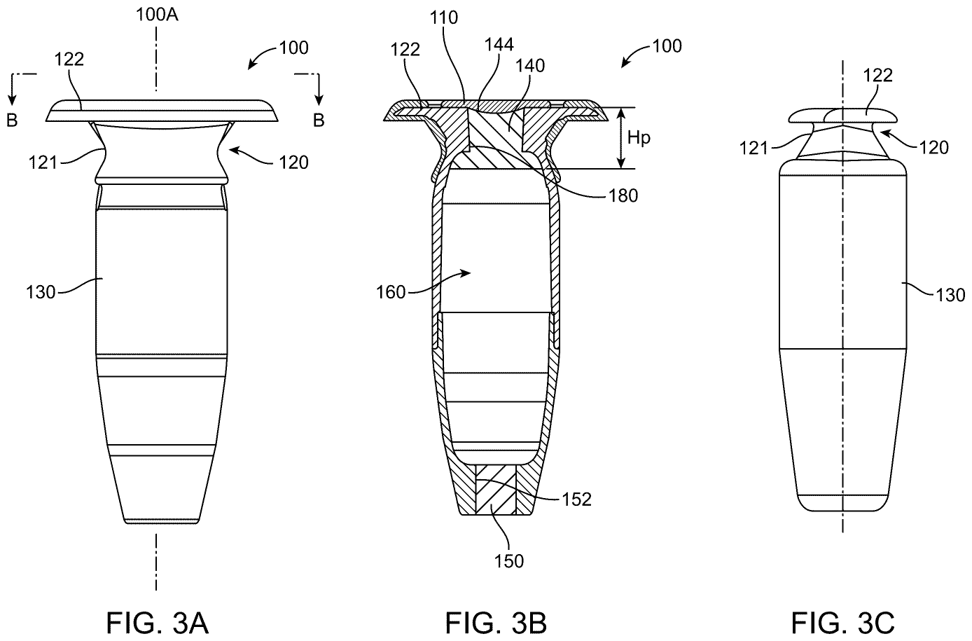

In a first implementation and as shown in FIGS. 3A-3C, the device 100 can include a housing body 130, a penetrable barrier or septum 140 and a porous structure 150. The body 130 can be a rigid, hollow refillable housing for implantation within an interior chamber of the eye, such as the posterior segment of an eye through a penetration in the sclera of the eye. The body 130 need not be rigid and can be pliable so as to expand upon filling, for example, as described in U.S. Pat. No. 11,419,759, which is incorporated by reference herein. The body 130 has a proximal end region and a distal end region. The body 130 has an inner surface that defines, at least in part, a reservoir 160 for holding a therapeutic material or agent(s) (see FIG. 3B). The septum 140 can be positioned within a proximal end region of the body 130 such as within an opening or bore 180 in an access portion of the device that leads into a reservoir 160 of the device. The porous structure 150 can be positioned within another region of the body 130 a distance away from the septum 140 such as within an opening 152 leading out of the reservoir 160 of the device. For example, the porous structure 150 can be positioned near a distal end region of the body 130 opposite the location of the more proximal septum 140. The reservoir 160 can have a volume sized to deliver therapeutic amounts of therapeutic agent to the eye for an extended period of time and the porous structure 150 can be configured to release therapeutic agent contained within the reservoir 160 over the extended period of time. The body 130 can include a proximal retention structure 120 including an extrascleral flange 122 that projects from the proximal end region of the body 130. The extrascleral flange defines an access portion opening or bore 180 that extends from an upper surface of the flange 122 into the reservoir 160 defined by the body 130. The septum 140 can be positioned, at least in part, within the bore 180 such that it forms a seal with the proximal end region of the body 130.

As will be described in more detail below, the devices described herein can also include an elastomeric encasement 110 extending over at least the upper surface of the flange 122 and the upper surface of the septum 140 so as to aid in preventing displacement of the septum 140 relative to the bore 180 upon penetration by needle. The encasement 110 can encapsulate the upper surface and the lower surface of the flange 122 without bonding to the flange 122. The septum 140 can be retained within the device by at least two primary bonds. A first bond is formed between the curved outer surface 145 of the septum 140 and the bore 180 in the proximal body 130 of the device. The first bond between the curved outer surface 145 of the septum 140 and the bore 180 can be formed using an epoxy adhesive that can include an amine-based epoxy curing agent. The encasement 110 can bond only to the upper surface 144 of the septum 140 forming a second bond. The encasement 110 encapsulates the retention structure 120 and bonds to at least the upper surface 144 of the septum 140 of the device. The encasement 110 can be configured to improve the integrity of the septum 140 and its sealing engagement within the bore 180 for repeated injection and long-term implantation. The encasement 110 will be described in more detail below.

Again with respect to FIGS. 3A-3C and as mentioned above, a distal end region of the body 130 can include another opening 152, for example opposite the proximal bore 180 into the reservoir 160, that extends between the inside of the reservoir 160 out of the body 130. The porous structure 150 can be coupled to or positioned, at least in part, within the opening 152. It should be appreciated that the porous structure 150 can be coupled to or positioned within other regions besides the distal end region of the body 130. The porous structure 150 can be affixed within an opening 152 in distal end of body 130, for example with glue or other material(s). Alternatively, or in combination, the distal end of the body 130 can include an inner diameter sized to receive the porous structure 150, and the body 130 can include a stop to position the porous structure 150 at a predetermined location on the distal end so as to define a predetermined size of reservoir 160.

Still with respect to FIGS. 3A-3C, the reservoir 160 within the body 130 of the device 100 can extend along axis 100A between the septum 140 positioned proximally within the bore 180 distally to the location of the porous structure 150. Therapeutic formulations injected into device 100 can be released from the reservoir 160 in accordance with the volume of the reservoir 160 and a release characteristic or release rate index of the porous structure 150. The volume of the reservoir 160 can be sized to deliver therapeutic amounts of a therapeutic agent to the eye for an extended period of time. The volume of the reservoir 160 can be substantially determined by an inner cross-sectional area of the body 130, such as the distance between the proximal, septum 140 and the porous structure 150. The release rate index (RRI) can be used to determine the release of the therapeutic from the device 100. RRI encompasses (PA/FL) where P comprises the porosity, A comprises an effective area, F comprises a curve fit parameter corresponding to an effective length and L comprises a length or thickness of the porous structure 150. Additional details regarding release characteristics of the porous structure 150 that can be used in the various devices described herein can be found, for example, in U.S. Publication No. 2014/0033800, which is incorporated herein by reference in its entirety.

In some implementations, the body 130 can have a dimension such that its length generally exceeds its width or diameter. The body 130 can have a diameter sized within a range, for example, from at least about 0.5 mm to at least about 4 mm, from at least about 1 mm to at least about 3 mm. In some implementations the diameter of the body 130 at its widest point can be about 2.5 mm, for example. The body 130 can have a length sized so as to extend from the conjunctiva 16 to the vitreous 30 along axis 100A to release the therapeutic agent into the vitreous 30. The body 130 can have a length sized within a range, for example, from at least about 2 mm to at least about 14 mm, from at least about 4 mm to at least about 10 mm. In some implementations, the length of the body 130 from the upper-most surface to the distal-most surface can be about 8.5 mm, for example. The flange 122 can remain outside the sclera and have a thickness of about 0.25 mm to about 0.5 mm. The above dimensions are provided as example dimensions and are not intended to be limiting. It should be appreciated that a variety and combination of dimensions are to be considered herein. For example, the body 130 can be configured to expand and thus, the body 130 at its widest point upon filling can be larger than the dimensions described above.

The body 130 and reservoir 160 can each (although not necessarily both) have an elliptical or oval cross-sectional shape, for example. This elongation of the device along one direction can allow for increased drug in the reservoir 160 while at the same time decreasing interference in vision, for example, as the major axis of the ellipse can be aligned substantially with the circumference of the pars plana region 25 of the eye extending substantially around the cornea 12 of the eye, and the minor axis of the ellipse can be aligned radially with the eye so as to decrease interference with vision as the short axis of the ellipse extends toward the optical axis of the eye corresponding to the patient's line of sight through the pupil.

Although reference is made to an elliptical or oval cross-section, many cross-sectional sizes and shapes can be used such as circular, square or rectangular with a short dimension extending toward the pupil of the eye and the long dimension extending along the pars plana of the eye.

One or more regions of the body 130 of the devices described herein can be formed of a substantially rigid, biocompatible material. In some implementations, the walls of the body 130 including at least the proximal retention structure 120 down to and including the porous structure 150 are substantially rigid such that the reservoir 160 has a substantially constant volume when the therapeutic agent is released from the device so as to maintain a stable release rate profile, for example when the patient moves. The reservoir 160 can remain substantially rigid and have a substantially constant volume even during injection of the therapeutic agent into the device, for example a device already implanted in the eye.

The devices described herein need not be rigid and can instead include non-rigid walled reservoirs configured to enlarge following implantation such as by filling with treatment solution. The expandable reservoirs may be used with any of the various implementations of a device or system. Further, reference to an expandable reservoir can include a reservoir wall that is pliable and able to be folded, compressed, contracted, etc. into a low profile configuration that is suitable for insertion into the eye in a manner that minimizes the size of penetration. The wall of an expandable reservoir may be pliable or flexible, but need not be stretchy or elastomeric in order to enlarge in size to hold the treatment solution. The expandable reservoir can include a reservoir wall that tents, unfolds, expands, stretches, or otherwise enlarges the overall cross-sectional size of the reservoir compared to the low profile configuration suitable for insertion. It should be appreciated that the terms unfold, expand, enlarge, and other terms used to refer to this shape change of the reservoirs described herein may be used interchangeably.

One or more regions of the body 130, one or more regions of the retention structure 120 as well as other portions of the devices described herein, alone or in combination, can be formed of one or more of many biocompatible materials including, but not limited to materials such as acrylates, polymethylmethacrylate, siloxanes, metals, titanium stainless steel, polycarbonate, polyetheretherketone (PEEK), polyethylene, polyethylene terephthalate (PET), polyimide, polyamide-imide, polypropylene, polysulfone, polyurethane, polyvinylidene fluoride, polyphenylene polyphenylsulfone or PTFE, and others. Alternatively or in combination, one or more portions of the devices described herein, such as the body 130, can be formed at least in part from an optically transmissive material such that the body 130 can be translucent or transparent, such that when the device 100 is loaded with therapeutic agent the reservoir 160 can be visualized outside the patient prior to implantation, for example when injected with a formulation of therapeutic agent prior to implantation in the physician's office. The encasement 110 can also be a transparent or translucent material for visualization of the septum 140 of the device. This visualization of the reservoir 160 can be helpful to ensure that the reservoir 160 is properly filled with therapeutic agent by the treating physician or assistant prior to implantation. For example, transparency can enable visualization, for example, using an indirect ophthalmoscope, of the contents of the reservoir 160 of an implanted device allowing one to confirm that no air is trapped in the device and/or verify the clarity of the device contents. A cloudy appearance, for example, may indicate that some degree of contamination, microbial or otherwise, has occurred. The biocompatible, optically transmissive materials can include one or more of acrylate, polysulfone, polyacrylate, methlymethacrylate, polymethylmethacrylate (PMMA), polycarbonate, glass or siloxane.

The porous structure 150 can include one or more of a release control element, a release control mechanism, permeable membrane, a semi-permeable membrane, a material having at least one hole disposed therein, channels formed in a rigid material, straight channels, nano-channels, nano-channels etched in a rigid material, laser drilled holes, laser etched nano-channels, a capillary channel, a plurality of capillary channels, one or more tortuous channels, sintered material, sintered rigid material, sintered glass, sintered ceramic, sintered metal, sintered titanium, tortuous micro-channels, sintered nano-particles, an open cell foam or a hydrogel such as an open cell hydrogel. Porous structures considered herein are described in U.S. Pat. Nos. 8,399,006; 8,623,395; 9,033,911; and US Publication No. 2014/0033800, filed Nov. 10, 2011, the entire disclosures of which are incorporated herein by reference thereto.

Again with respect to FIGS. 3A-3C and as mentioned above, the retention structure 120 can protrude outward from the proximal end region of the body 130. At least a portion of the underside of the retention structure 120 can contact the sclera 24 and at least a portion of the upper side of the retention structure 120 can contact the conjunctiva 16. In some implementations, the retention structure 120 can be configured to contact the sclera 24 such that the retention structure 120 is at least partially embedded within the thickness of the sclera 24 and does not necessarily sit on an upper surface of the sclera or the conjunctiva. The retention structure 120 can have a thickness between the underside and the upper side that is between about 0.25 mm to about 0.5 mm.

The retention structure 120 can include a narrowed portion 121 and a wider, extrascleral flange 122 extending proximally from the narrowed portion 121. The narrowed portion 121 can have a cross-section sized to fit in an elongate incision or a puncture through the pars plana region 25 without causing gaping of the tissue near either end of the incision. For example, an incision can be made with a device having a straight, flat blade, for example a 3.2 mm blade. Penetrating the sclera with such a blade can result in exposed scleral tissue that may need to be sealed (e.g., 6.4 mm or 2×3.2 mm). A cross-sectional region of an implant positioned within the cut region of the sclera, for example having a perimeter of 6.4 mm and a diameter of about 2 mm, could open the wound such that there would be relatively large voids on either side of the device, for example about 2.2 mm between either side of the device and the farthest aspects of the exposed sclera. These voids can result in cut portions of the sclera remaining exposed and unsealed. The geometry of the narrowed portion 121 of the devices described herein can be designed to minimize the length of cut scleral tissue that remains exposed and/or unsealed.

The narrowed portion 121 can have a first cross-sectional distance across, or first dimensional width, and a second cross-sectional distance across, or second dimensional width, in which the first cross-sectional distance across is greater than the second cross-sectional distance across providing the narrowed portion 121 with an elongate cross-sectional profile. The elongate cross section of the narrowed portion 121 can be sized in many ways to fit the incision. The elongate cross section can have a first dimension longer than a second dimension and can have one or more of many shapes such as dilated slit, dilated slot, lentoid, oval, ovoid, or elliptical. It should also be appreciated that the narrowed portion 121 can have other cross-sectional shapes, for example, a circular shape, if desired. The dilated slit shape and dilated slot shape can correspond to the shape assumed by the scleral tissue when cut and dilated. The lentoid shape can correspond to a biconvex lens shape. The elongate cross-section of the narrowed portion 121 can include a first curve along a first axis and a second curve along a second axis that is different than the first curve. The narrowed portion 121 can be sized and configured to receive the sclera 24 upon implantation in the eye 10 when the flange 122 is positioned between the sclera 24 and the conjunctiva 16 and the distal end of the body 130 extends into the vitreous 30.

Flange 122 of the retention structure 120 can include a first distance across and a second distance across. The first distance across can be greater than the second distance across (see FIGS. 3B and 3C, for example). The first distance across can result in the flange 122 having a diameter greater than a largest diameter of the body 130 (see e.g., FIG. 3B) whereas the second distance across can result in the flange 122 having a diameter equal to or less than a largest diameter of the body 130 (see e.g., FIG. 3C). The flange 122 can have a variety of shapes, such as rectangular, square, oval, elliptical, circular, teardrop, polygonal or other shape. The flange 122 can be formed as a smooth protrusion configured for placement along a portion of the sclera 24. In some implementations, the flange 122 is positioned under the conjunctiva 16, such that the conjunctiva 16 covers and protects the device 100.

Coverage of the flange 122 by the conjunctiva 16 can also aid in preventing infection by providing a barrier. The flange 122 can be formed from a translucent material such that the physician can visualize tissue under the flange 122 to assess the patient and to decrease appearance of the device 100 when implanted.

As mentioned above, the septum 140 can be positioned, at least in part, within bore 180 sealing the reservoir 160 on a proximal end region of the device 100 (see FIG. 3B). The septum 140 can be a septum configured to receive and be repeatedly penetrated by a sharp object such as a needle for injection of the therapeutic agent into the reservoir 160. The septum 140 can be configured to re-seal when the sharp object is removed. The septum 140 can be a pre-molded soft, high strength material. The septum 140 need not be pre-molded to the exact shape and size of the bore 180 within which it is to be positioned. Preferably, the septum 140 is slightly oversized relative to the bore 180 such that upon positioning of the septum 140 within the body 130, the inner surfaces of the bore 180 apply an amount of radial compression on the pre-molded septum 140. The body 130 defining the bore 180 can be formed of a higher durometer material compared to the material of the septum 140 such that the smaller dimension of the higher durometer body 130 applies compression and provides additional septum seal performance, such as by encouraging re-sealing of the septum 140 after penetration and removal of a needle track during refilling of the reservoir. In some implementations, the septum 140 can be formed from one or more elastic materials such as siloxane, rubber, or another liquid injection molding silicone elastomer such as NUSIL MED-4810 (NuSil Silicone Technology, Carpinteria, CA), or other elastomer such as polyurethane elastomers (thermoplastic urethanes) or polyurethane/siloxane copolymers. In some implementations, the septum 140 can include an opaque material and/or a colored material such that it can be visualized by the treating physician.

As described above and as best shown in FIGS. 3A-3C, the septum 140 can be positioned within a proximal end region of the body 130 at least in part within the bore 180 of the access portion. As such, the overall shape of the external surface of the septum 140 can correspond generally to the shape of the surface(s) near the bore 180 against which the septum 140 contacts to mate and seal. It should be appreciated that the points of contact between the septum 140 and the body 130 can vary. The septum 140 can make contact, for example sealing contact, with at least one or more surfaces or regions of the upper end of the reservoir chamber, the body 130, the retention structure 120, the narrowed portion 121, the flange 122, the bore 180, and/or a combination thereof.

The septum 140 can have an upper surface 144, a middle region 145 formed by a curved outer surface, and a lower surface 142 on a distal end region 143 of the septum 140. The upper surface can be sized to reside within and mate with at least a portion of the flange 122, such as an upper end of the bore 180. The upper surface of the septum 140 can be available through the access region opening or bore 180 of the device. The middle region 145 can be sized to reside within and mate with inner surfaces of the narrowed portion 121 of the retention structure 120 defining the bore 180. The middle region can be a reduced diameter region or form a “waist” in the septum 140.

In some implementations, the distal end region 143 can have a diameter that is the same as or greater than the narrowed portion 121 of the retention structure 120. For example, the distal end region 143 of the septum can be formed by one or more tabs, a flared skirt, flange, rib or other feature of enlarged diameter compared to the middle region 145 and/or upper surface 144 sized to reside within and mate with at least a portion of the retention structure 120 located distal to the narrowed portion 121 and/or an upper region of the reservoir 160 such as with an inner wall of the body 130. The distal end region 143 of the septum 140 is configured to contact an inner wall surface near the upper end of the reservoir 160 that surrounds the bore 180 of the access portion (see FIG. 3B). The features of the distal end region 143 having an enlarged diameter compared to the middle or upper regions of the septum 140, such as the one or more tabs, flanges, or flared skirt, can also aid in retaining the septum 140 within the bore 180 during manufacturing. The cross-sectional diameter of the septum 140 distal to the middle region 145 in at least a first direction can be equal to, more or less than the cross-sectional diameter of the upper surface 144.

Repeated injection as well as long-term implantation of the device 100 can affect the integrity of the septum 140. For example, repeated injection through the septum 140 can at least partially damage the device and negatively affect the seal between the inner surfaces of the body 130 and the outer surfaces of the septum 140. Further, over time after implantation the septum 140 can loosen relative to the body 130. Described herein are features to improve the integrity of the septum 140, its sealing engagement with the bore 180 of the body 130 and/or retention structure 120, and the effectiveness of the access region for repeated injection and long-term implantation of the re-fillable devices described herein.

As mentioned above, the devices described herein can be coupled to a encasement 110 configured to improve the integrity of the septum 140 and its sealing engagement with the bore 180 for repeated injection and long-term implantation. This provides a benefit to a device intended to be implanted long-term and re-filled while implanted, such as those described herein. The encasement 110 is coupled to at least a proximal end region of the device so as to encapsulate the retention structure 120 and bond to the upper surface of the septum 140. For example, the encasement 110 can couple to at least a portion of a proximal end region of the device 100, including one or more combinations of the upper surface of the septum 140 positioned within the opening of the access portion bore 180, an upper surface of the proximal retention structure 120 including the flange 122, a lower surface of the proximal retention structure 120 including the flange 122, the narrowed portion 121 of the retention structure 120, and at least a portion of an outer surface of the body 130 near the proximal end region.

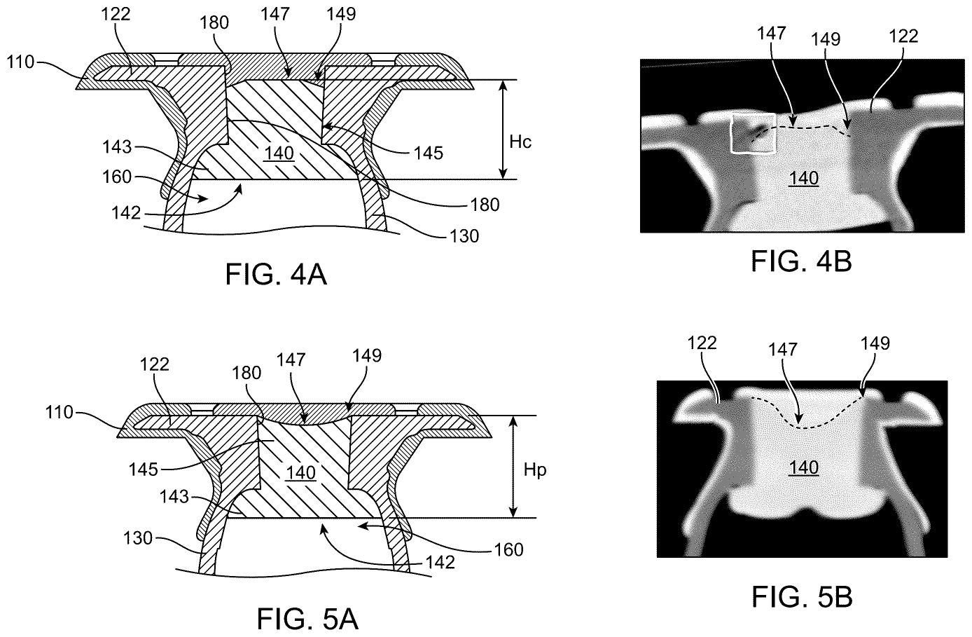

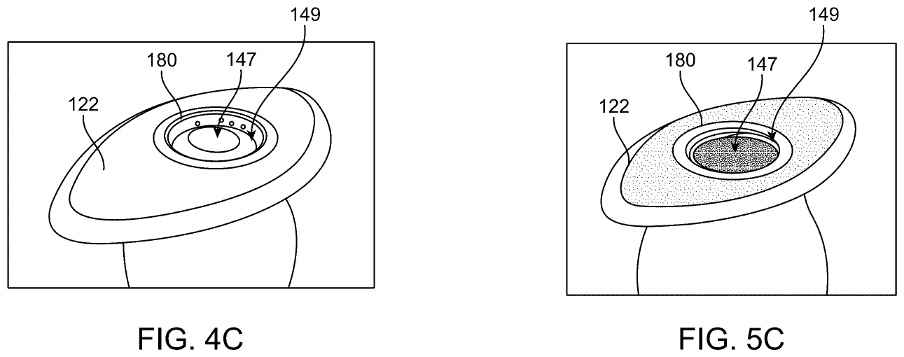

The encasement 110 and the proximal retention structure 120 (or any other region coupled to the encasement 110 such as the flange 122), can have corresponding shape profiles. The thickness of the over-molded encasement 110 can vary from approximately 0.007″ (0.180 mm) to approximately 0.025″ (0.640 mm). In some implementations, the septum 140 had a concave or conical shaped upper surface in which a central portion of the septum 140 lies below the outer perimeter of the septum 140 forming a depression within the central portion. The thickness of the over-molded encasement 110 in the region of the depression can exceed 0.025″ without significantly increasing the effective thickness of the flange 122 or shape profile of the extrascleral portion of the device. The encasement 110 can extend beyond the outer diameter of the flange 122 as best shown in FIGS. 4A and 5A. The encasement 110 can also extend upward from the upper surface of the flange 122 and provide a slightly thicker and slightly higher profile to the access portion under the conjunctiva. During injection of the therapeutic agent into the reservoir 160, the needle can extend through the encasement 110 as it penetrates the septum 140. The encasement 110, like the septum 140, can be configured to re-seal when the needle or other sharp object is withdrawn.

The proximal retention structure 120 can include one or more through-holes, apertures, indentations or other features. The flange 122 can include one or more apertures extending therethrough. Upon application of the encasement 110, the apertures create mechanical struts of the over-molded encasement material that extend through one or more regions of the flange 122. The mechanical struts of over-molded encasement material provide some anchoring support as well as facilitate good filling of the over-mold. The apertures can also allow for a thin, uniform layer of over-mold material to form on the underside of the flange 122 or other another region of the retention structure during over-molding. It should be appreciated, however, that mechanical struts of the over-molded material can be formed by over-molded material extending only partially through apertures in the flange 122. Further, instead of apertures, the flange 122 can include only partial-thickness holes or indentations in the flange 122. The indentations can be on an external surface of the flange 122 such as in the upper and/or lower surfaces of the flange 122. The external surfaces of the flange 122 can also be textured such that the over-molded material of the encasement 110 can penetrate and fill additional indented regions of the flange 122 to provide a better coupling between the flange 122 and the material of the encasement 110.

The proximal end region of the body 130 of the therapeutic device 100 can be machined from a piece of material, or injection molded, so as to form the retention structure 120, flange 122 and/or the narrowed portion 121. As described above, the septum 140 can be pre-molded and the encasement 110 can be over-molded. Alternatively, the encasement 110 can be pre-molded and bonded to the pre-molded septum 140. The septum 140 and encasement 110 can be the same material and over-molded around the flange 122 using a single step injection molding process. Alternatively, the septum 140 or encasement 110 can be two different materials and over-molded around the flange and cured in two independent steps.

The septum 140 preferably bonds along its waist, or the curved outer surface of the middle region 145 connecting the upper surface 144 to the lower surface 142 of the septum 140, to the inner surfaces of the bore 180 of the body 130. The septum 140 bonds to the encasement 110 along its upper surface. The encasement 110 can bond only to the septum 140 although it may encapsulate the flange 122 of the body 130. The proximal end region of the body 130 can be any of a variety of materials such as polysulfone. The septum 140 positioned within the proximal end region of the body 130 can be pre-molded, soft, high-strength material such as a liquid injection molding silicone elastomer such as MED-4810 (NuSil Silicone Technology, Carpinteria, CA). The encasement 110 can be an over-molded, high durometer material such as a translucent, liquid silicone rubber like MED-4880 or MED-4860 (NuSil Silicone Technology, Carpinteria, CA).

FIGS. 4A-4B show an implementation of a device in which the bore 180, the septum 140 and the retention structure 120 are over-molded by the encasement 110. The encasement 110 encapsulates the upper surface of the proximal retention structure 120 and the lower surface of the proximal retention structure 120. The upper surface of the septum 140 is accessible from the proximal end region of the device to as to bond to the encasement 110. As discussed elsewhere herein, the septum 140 can be a pre-molded element having a first shape that is installed within the bore 180 at the proximal end region of the device and cut to a second shape following positioning within the bore 180. Trimming the septum 140 to a second shape exposes an interior of the septum 140 maximizing the surface area available for improved bonding. The curved outer surface of the middle region 145 of the septum 140 engages with the inner surface of the body 130 forming the bore 180. In the implementation shown in FIGS. 4A-4C, the septum 140 installed within the bore 180 is trimmed only at the central region 147 and is left untrimmed at the outer perimeter region 149. The trimmed central region 147 can be substantially planar and the untrimmed outer perimeter region 149 lies below the plane of the central region 147. The entire septum 140, including the planar central region 147 and the beveled outer perimeter region 149 lying below the plane of the central region 147, lies below the plane of the upper surface of the flange 122 of the retention structure 120 such that the septum 140 is countersunk relative to the flange 122. The interface between the encasement 110 and the septum 140 is less than a full diameter of the septum 140 and relies upon the substantially planar contact area with the central region 147. The septum 140 can be placed under tension prior to trimming to achieve the second shape.

The septum 140 positioned into a proximal region of the device 100 can be maintained in the bore 180 in an adhesion-free manner and rely on the mating features between the external surface of the septum 140 with the corresponding surfaces of the bore 180 against which the septum 140 abuts and seals. Preferably, the septum 140 is adhered within the bore 180 of the retention structure 120, such as by a two-part epoxy. Two-part epoxies include a Part A resin and a Part B hardener. Amine-based curing agents may be used in the epoxy hardener. Platinum catalysts can be susceptible to poisoning from amine-based epoxy curing agents.

Thus, the epoxy fixation between the septum 140 and the body 130 can impair the bonding between the septum 140 and the encasement 110.

Again with respect to FIGS. 4A-4C, the countersunk septum 140 can create gaps near where the beveled outer perimeter region 149 lying below the plane of the central region 147 meets the inner surfaces of the bore 180. For example, the maximum height Hc between the lower surface 142 of the septum 140 and the planar upper surface of the central region 147 is greater than the height Hp of the septum 140 between the lower surface 142 and the outermost edge of the perimeter region 149. While the planar upper surface of the central region 147 may extend clear to the upper surface of the flange 122, the shorter height Hp of the outer perimeter region 149 forms a sunken region of the septum 140 within the bore 180. This sunken region creates a gap or an upper region of the bore 180 that is not bonded to the curved outer surface 145 of the septum 140. This gap can collect and trap epoxy used to bond the septum 140 to the body 130. The excess epoxy in this region can impair or prevent bonding between the upper surface of the septum 140 and the elastomeric encasement 110.

FIGS. 5A-5C show another implementation of a device in which the bore 180, the septum 140 and the retention structure 120 are over-molded by the encasement 110. The septum 140 is pre-molded to a first shape and cut to a second shape once the septum 140 is positioned within the bore 180. Trimming the septum 140 to the second shape exposes an interior of the septum 140. The exposed interior spans across the full upper surface of the septum 140. The septum 140 installed within the bore 180 at the proximal end region of the device is trimmed to provide a septum/encasement interface that spans the full diameter of the septum 140 thereby eliminating any gaps or sunken region at an upper region of the bore 180 that might trap epoxy between the encasement 110 and the septum 140. The curved outer surface 145 of the septum 140 in FIGS. 5A-5B engages with the inner surface forming the bore 180 up to or near a level of the upper surface of the flange 122 minimizing or eliminating the sunken region that can trap epoxy.

The entirety of the upper surface of the septum 140 is trimmed including the trimmed central region 147 and the trimmed outer perimeter region 149. The trimmed central region 147 is concave or conical in shape prior to installation of the encasement 110 forming a depression in the upper surface of the septum 140. The concave or conical shape increases the surface area available for the encasement 110 and the septum 140 to bond compared to that provided by the planar central region 147 in the embodiment shown in FIGS. 4A-4B. The material of the encasement 110 can fill the depression in the upper surface of the septum 140. A portion of the outer perimeter region 149 forming a proximal-most surface of the septum 140 lies above a plane of the central region 147 so that the central region 147 of the upper surface of the septum lies below the perimeter region 149. While the central region 147 lies below the plane of the upper surface of the flange 122 of the retention structure 120, the outer perimeter region 149 of the upper surface of the septum lies flush or substantially flush with the plane of the upper surface of the flange 122. The central region 147 can be countersunk, but the outer perimeter region 149 is not counter sunk relative to the flange 122. In this implementation, the interface between the encasement 110 and the septum 140 is across the full diameter of the septum 140 and includes an increased contact area of both the outer perimeter region 149 and the central region 147. The contact area at the interface is non-planar (e.g., concave depression). The outer perimeter region 149 lying substantially flush with the plane of the upper surface of the flange 122 minimizes or eliminates any gaps between the outer perimeter region 149 and the inner surfaces of the bore 180. The maximum height Hp of the septum 140 between the lower surface of the septum 140 to the upper surface of the outer perimeter region 149 can be greater than the height Hc of the septum 140 between the lower surface and the upper surface of the central region 147. The maximum height Hp can be greater than about 1.05 mm and less than about 1.35 mm. The epoxy bond between the curved outer surface of the septum and the bore 180 covers the entire surface of the bore 180 up to or near a level of the upper surface of the flange 122 compared to that of FIGS. 4A-4B in which the epoxy bond covers only a partial portion of the bore 180. Additionally, no gaps exists at the outermost edge of the perimeter region 149 that can collect and trap epoxy, thereby improving the bond between the material of the septum 140 and the material of the encasement 110. The larger trimmed surface of the septum 140 for bonding to the encasement 110 improves the bond between the material of the septum 140 and the material of the encasement 110.

FIG. 4B is a CT image of a proximal end region of a device having a septum 140 with an untrimmed outer perimeter region 149 that is lying below the plane of the upper surface of the flange 122 creating a sunken region or a gap within which epoxy can collect. The image illustrates separation (see square drawn in FIG. 4B) that occurred due to poor bonding between the outer perimeter region 149 of the septum 140 and the encasement 110. The encasement 110 has lifted away from the beveled outer perimeter region 149 lying below the plane of the flange 122 and remains bonded only at the central region 147. FIG. 5B is a CT image of a proximal end region of a device having a fully trimmed upper surface of the septum 140 from the outer perimeter region 149 to the central region 147. The outer perimeter region 149 lies substantially flush with the plane of the upper surface of the flange 122 and the central region 147 is countersunk into a concave or conical shape below the plane of the flange 122. The encasement 110 is fully bonded across the entire span of the septum 140 available through the bore 180 of the device.

FIG. 4C is another CT image of the proximal end region of a device without the encasement 110 present. The septum 140 is visible within the bore 180 of the device. The central region 147 is trimmed into a concave or conical shape as opposed to a planar surface. However, the septum 140 includes an outer perimeter region 149 that is untrimmed and lies below the plane of the upper surface of the flange 122. FIG. 5C is a CT image of the proximal end region of a device also without a encasement 110 present. The septum 140 is visible within the bore 180 and shows a fully concave shape over the central region 147 such that the outer perimeter region 149 forms an outermost edge of the septum 140 that lies substantially even with the upper surface of the flange 122 so that any gaps in this outer perimeter region 149 are eliminated.

A two-part epoxy can be used to form the first bond between the septum 140 and the proximal body 130 of the implant device. The geometry of the septum 140 relative to the bore 180 improves retention of the septum 140 over time and upon repeated penetrations by mitigating inadvertent exposure of the silicone to the epoxy. The septum 140 can be a silicone material and the proximal body 130 defining the bore 180 can be a material such as polysulfone. Residual materials from the hardener or Part B of the epoxy (e.g., Trimethyl-1,6-hexanediamine, TMHMD) can interfere with silicone polymerization needed to bond the silicone material of the septum 140 to the silicone material of the encasement 110. TMHMD residue can persist in septum/proximal body subassemblies prior to overmolding with the encasement 110, even if no traces of TMHMD are found in the finished implant. As will be described in more detail below, in addition to the geometry of the septum 140 relative to the bore 180 discussed above, additional curing steps of the septum/proximal body subassemblies (i.e., the septum 140 assembled and adhered within bore 180 of the proximal body 130 of the implant device 100) improves the bond strength between the septum 140 and the encasement 110 by vaporizing and removing residual TMHMD from the subassemblies prior to assembly and bonding with the encasement 110.

The curved outer surface of the septum 140 forms a first bond with the bore 180 of the body 130 using an epoxy adhesive, which can include an amine-based epoxy curing agent. The first bond is substantially free of TMHMD prior to forming the second bond between the encasement 110 and the upper surface of the septum 140. The first bond between the curved outer surface of the septum 140 and the bore 180 if the body 130 is cured in at least two stages prior to forming the second bond between the encasement 110 and the upper surface of the septum 140.

The first stage of curing the septum/proximal body subassembly includes heating the first bond between the septum 140 adhered within the bore 180 of the body 130 to about 67° C. for at least 1 hour up to about 3 hours. The second stage of curing includes heating the first bond to at least 115-135° C. for more than 30 minutes and less than about 4 hours, preferably about 3 hours. The bond strength between the septum 140 and the encasement 110 is greatly improved by the second curing stage of the septum 140 to the body 130 because the second curing stage eliminates the TMHMD residue in the epoxy that impairs the bond between the septum 140 and the encasement 110.

The overall robustness of the device is increased by increasing septum height relative to the bore within which it is bonded and increasing the trimmed septum surface area available for bonding to the encasement to eliminate gaps at the outer perimeter region 149 of the septum 140 and minimizing the epoxy contamination. The overall robustness of the device is also increased by reducing residual TMHMD from the septum/proximal body subassembly prior to overmolding with the encasement 110 by adding a second stage of epoxy curing that is performed at a temperature of at least 115° C. and for greater than 0.5 hour and less than about 4 hours.

It should be appreciated the residual TMHMD may be removed in other ways besides oven heating for curing, such as using solvent cleaners or reactive gas. Reducing the amount of epoxy dispensed to bond the septum to the proximal body can aid in reducing the residual TMHMD found in the constructs. For example, the epoxy dispensed within the bore 180 (or dispensed onto the curved outer surface of the septum 140) can be less than about 0.30 uL, less than about 0.25 uL, less than about 0.20 uL, or less than about 0.15 uL down to about 0.05 uL. Reducing the epoxy dispensed can minimize excess epoxy and its adverse impact on overmold/septum bonding.

Certain surface treatments can also be used during manufacturing of the devices described herein to enhance bonding between various components, including for example but not limited to, bonding primer agents such as NUSIL MED 161 or other surface activation techniques such as plasma treatment of the surfaces to be bonded. For example, the bore 180 within which the septum 140 is positioned can undergo plasma treatment. Air plasma treatment of about 3 seconds up to about 5 seconds can be used on the septum 140 while rotating the septum for the treatment duration.

### Refill Exchange

Initial filling of the device 100 with one or more therapeutic agents can occur prior to insertion or after insertion of the device 100 in a patient's eye. The septum 140 as well as the encasement 110 can be penetrated with a needle or access device attached to a syringe or injection device containing therapeutic agent.

The encasement 110 and the septum 140 can be penetrated during filling and/or refilling of the reservoir 160. The needle or access device can be inserted through the septum 140 until a distal opening of the needle enters the reservoir 160. The contents of the syringe or injection device can be injected into the reservoir 160 and the needle or access device can be removed from the septum 140.

The encasement 110 and the septum 140 can be configured to reseal after penetration during filling and/or refilling of the reservoir 160. The septum 140 can reseal around the path created by the needle or access device upon its removal. The device 100 also can be periodically refilled with therapeutic agent following surgical placement as needed by accessing the implanted device 100 and without necessitating device removal. The conjunctiva 16 can be lifted or incised away. Alternatively, the conjunctiva can be pierced with the needle or access device used to fill the device 100 such that a single penetration is performed through each of the conjunctiva 16, encasement 110, and septum 140. Once the needle or access device is inserted and located at the appropriate depth within the reservoir 160, injection of fresh therapeutic solution or exchange of pre-existing reservoir contents with fresh therapeutic solution can take place.

The therapeutic devices described herein can be implanted in the eye to treat the eye for as long as is helpful and beneficial to the patient. For example, the device can be implanted for at least about 1 month, 2 months, 3 months, 6 months, 1 year, 2 years, 3 years, 4 year, 5 years and up to permanently for the life of the patient. Alternatively. or in combination, the device can be removed when no longer helpful or beneficial for treatment of the patient. In other implementations, the device can be implanted for at least about 4 years to 10 years, for example a duration of treatment period for a chronic disease such as diabetic macular edema or age-related macular degeneration. The device can be periodically refilled in the physician's office with new therapeutic agent as indicated by disease progression. For diseases such as age-related macular degeneration, the device can be refilled as frequently as once every week, bi-weekly, monthly, bi-monthly, every 3 months, every 4 to 6 months, every 3 to 9 months, every 12 months, or any other period as indicated to treat a disease.

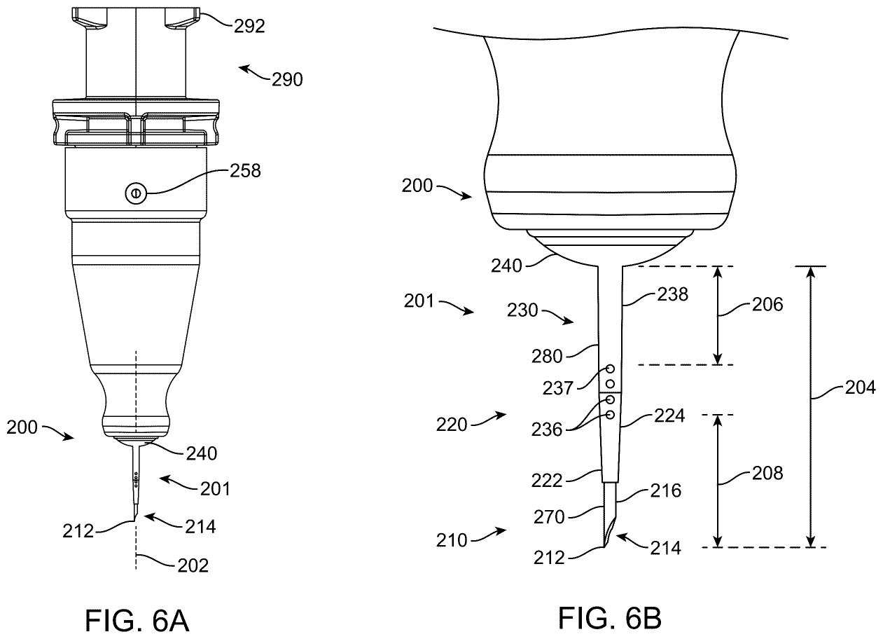

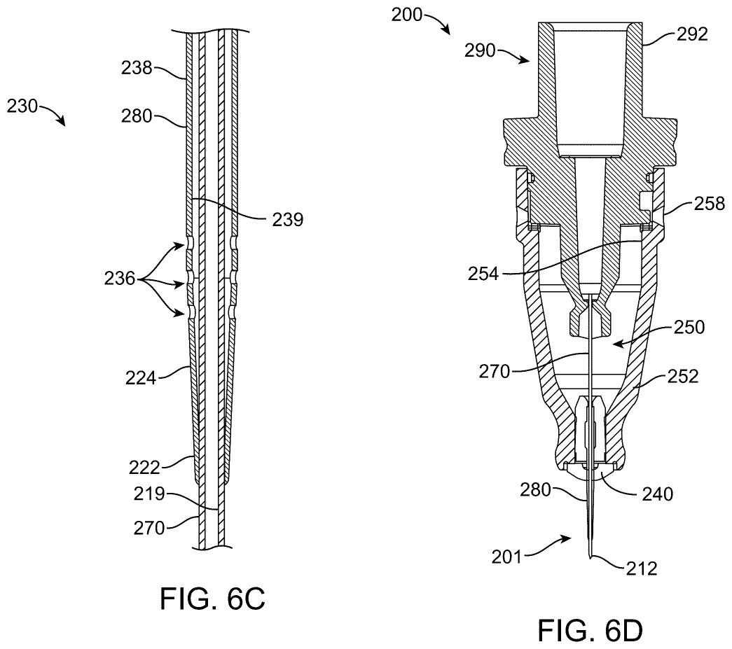

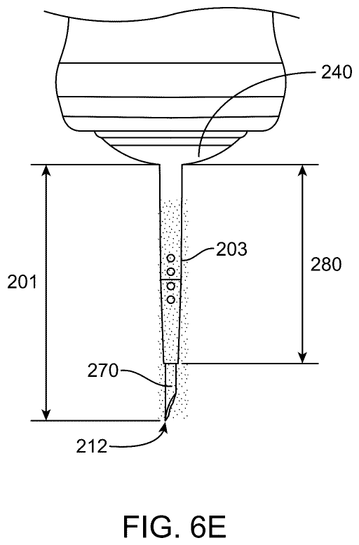

FIG. 6A is a side view of an exchange needle device 200 for refilling of an implanted therapeutic device 100 and FIG. 6B is a detail view of an elongate needle device 200 of FIG. 6A. FIG. 6C is a cross-sectional view of a segment of the elongate needle structure 201 of FIG. 6B. The device 200 can be coupled to or include on a proximal end region a syringe having a container to inject a therapeutic fluid into the reservoir 160 of the device 100. The device 200 can include an elongate needle structure 201 having a working length projecting from a proximal hub or connector. The working length of the needle structure 201 can be placed substantially within at least a portion of the device 100. The needle structure 201 can include at least one opening 214 to place the therapeutic fluid in the reservoir 160 of the device 100 and a plurality of side ports 236 to receive the fluid from the reservoir 160 of the implantable device 100.

Still with respect to FIGS. 6A-6C, the needle structure 201 can have a distal portion 210, an intermediate portion 220, and a proximal portion 230.

The distal portion 210 can include a distal tip 212 configured to penetrate the septum 140 of the implantable device 100. The at least one opening 214 to inject therapeutic fluid into the implantable device 100 can be found at or near the distal tip 212. The intermediate portion 220 can include a tapered section 224 that gradually increases a size of the channel formed in the septum 140 when the needle structure 201 is advanced through the septum 140 so as to maintain integrity of and mitigate damage to the septum 140. The tapered portion 224 can extend along axis 202 and can be without holes so as to decrease pressure to the septum 140 that may otherwise occur near the edge of a hole.

The needle structure 201 can include at least one opening 214 to place the therapeutic fluid in the reservoir 160 of the device 100 and a plurality of openings or side ports 236 extending through a wall of the outer cannula 280 to receive the fluid from the reservoir 160 of the implantable device 100 into an annular space between the inner and outer cannulae. The distal end of the inner cannula 270 can include a sharpened beveled tip forming the opening 214.

The proximal portion 230 can include the plurality of side ports 236 to receive the fluid from the reservoir 160 of the implantable device 100. The needle structure 201 can include a stop 240 to limit a depth of insertion of the needle structure 201 into the reservoir 160 of the device 100, for example, no greater than about 4.5 mm. The stop 240 can be a deformable material to engage with the tissue during injections. The proximal portion 230 can include an extension 238 extending from the stop 240. The extension 238 can be without holes to inhibit leakage when the fluid is exchanged and the stop 240 engages the conjunctiva 16.

When coupled to the therapeutic device 100, the stop 240 can be positioned to engage the conjunctiva 16 and the needle structure 201 can extend through the conjunctiva 16 and the septum 140 into the device 100. The needle structure 201 can be sized so as to place the distal tip 212 at a location within the device 100 when the surface of the stop 240 contacts the conjunctiva 16, for example. The distal tip 212 can be located on the needle structure 201 so as to place the distal tip 212 at a location from the septum 140 within the device 100 that is no more than a desired length, such as about ¾ of the length of the implantable device 100, and in some implementations no more than about half of the distance of the device 100. The extension 238 can extend substantially through the septum 140, for example, at least about half-way through the septum 140 so as to position the plurality of openings away from an external surface of the septum 140 and to inhibit leakage.

FIG. 6B shows a detail view of the needle structure 201 of the exchange needle device 200 of FIG. 6A having a working length projecting from the proximal hub. The needle structure 201 extends along axis 202 between the distal tip 212 and the stop 240. The distal portion 210 can include an extension 211 having a substantially constant cross-sectional size extending between the tip 212 to penetrate tissue and the intermediate portion 220. The needle structure 201 can include an inner cannula 270 (which may be referred to herein as an elongate tube or a fill cannula or a needle) extending distally from a proximal hub or connector. The inner cannula 270 has an outer surface, a proximal end, a distal end, and an inner surface defining a bore that extends to a distal opening at a distal end of the inner cannula 270. The elongate needle structure 201 also includes an outer cannula 280 or sheath extending distally from the proximal hub. The outer cannula 280 also includes an outer surface, a proximal end, a distal end, and an inner surface defining a bore extending to a distal opening at the distal end of the outer cannula 280. The inner cannula 270 can extend through the bore or internal lumen of the outer cannula 280 such that the outer cannula 280 surrounds at least a proximal end region of the inner cannula 270. The inner diameter of the outer cannula is sized relative to the outer diameter of the inner cannula 270 so as to form an annular space between the outer surface of the inner cannula 270 and the inner surface of the outer cannula 280. The distal end of the outer cannula 280 is located a distance proximal to the distal opening of the inner cannula 270. The elongate needle structure 201 of the exchange needle device 200 can additionally incorporate a lubricious coating on the outer surface of the outer cannula 280 and the outer surface of the inner cannula 270 extending distal to the distal end of the outer cannula 280. The lubricious coating covers at least a portion of the working length of the elongate needle structure 201.

The distal tip 212 of the inner cannula 270 has at least one opening 214 through which material injected through the lumen of the inner cannula 270 may flow out the opening 214. The distal tip 212 can be sharp or blunted. The outer cannula 280 can be configured to receive preexisting material from the reservoir 130 such that it can be flushed out from the reservoir 160 upon filling with new material through the inner cannula 270. Thus, the outer cannula 280 can include at least one opening into its lumen. The opening can be formed at a distal end of the outer cannula 280 formed between the outer surface of the inner cannula 270 and the inner surface of the outer cannula 280. Alternatively, or in additionally, the opening can include a plurality of openings or side ports 236 through a wall of the outer cannula 280 as described in more detail below.

Still with respect to FIG. 6B, the extension 211 can include a portion of the inner cannula 270 extending from the stop 240 to the tip 212 of the inner cannula 270. The tip 212 can be configured to penetrate tissue, such as the tip of the needle to penetrate conjunctival tissue. The tip 212 and the opening 214 can be located a distance 204 from the stop 240 and the plurality of side ports 236 to provide efficient exchange of the fluid within the reservoir 130 of the implanted device 100.

In some implementations, the opening 214 is placed at a distance from the stop 240 greater than the plurality of side ports 236 such that the opening 214 is located distal to the plurality of side ports 236. This relative position between the opening 214 and the plurality of side ports 236 can inhibit mixing of the injected therapeutic fluid moving into the reservoir 160 through opening 214 with the fluid within the implanted device 100 moving out of the reservoir 160 through side ports 236. The opening 214 can be separated from the plurality of side ports 236 by a distance 208, such that the opening 214 can be located below the plurality of side ports 236 when the therapeutic fluid is injected.

The therapeutic fluid may have a density greater than the fluid of the implanted device and opening 214 can be placed below the plurality of side ports 236 when the therapeutic fluid is injected to inhibit mixing of the fluids (i.e., the fluid moving in from the fluid moving out). The axis 100A of the implantable device 100 and the corresponding axis of the reservoir 160 can be oriented away from horizontal, such that porous structure 120 may be located below the septum 140 when the therapeutic fluid is injected. The axis 202 can be oriented away from horizontal such that opening 214 can be placed below the plurality of side ports 236. The therapeutic fluid having the greater density can flow toward the distal end of the therapeutic device and the displaced fluid from the implantable device having the lesser density can be received by the plurality of side ports 236 located above the opening 214. It should be appreciated that inner cannula 270 can be movable relative to the outer cannula 280 or the two components can be in a fixed configuration relative to one another.

Examples of therapeutic agents and corresponding formulations and fluids that may be delivered using the devices described herein are described in the applications incorporated by reference herein and in Table 1 of U.S. application Ser. No. 14/937,784, published as U.S. 2016/0128867, which is incorporated herein in its entirety. Therapeutic agents that can be delivered from the devices described herein include but are not limited to triamcinolone acetonide, bimatoprost or the free acid of bimatoprost, latanoprost or the free acid or salts of the free acid of latanoprost, ranibizumab, travoprost or the free acid or salts of the free acid of travoprost, faricimab, zifibancimig, timolol, levobunalol, brimonidine, dorzolamide, brinzolamide, and others for the treatment of ocular diseases.

The therapeutic agents and corresponding formulations can have a density greater than the density of the fluid within the chamber of the implanted device. For example, one or more of the therapeutic agent or a stabilizer can increase the density of the therapeutic fluid. In many embodiments the therapeutic fluid having the greater density comprises a stabilizer, such as trehalose, and the therapeutic agent such as a protein comprising an antibody fragment. Alternatively or in combination, the therapeutic formulation can include an amount of therapeutic agent sufficient to provide a density greater than the fluid of the implanted device. The difference in density can be within a range from about 1% to about 10% and can depend on the density of the fluid within the reservoir chamber of the therapeutic device and density of the therapeutic fluid placed in the reservoir chamber with the exchange apparatus. The density of the therapeutic fluid may correspond to a density of the therapeutic agent and a density of the stabilizer (when present). In many embodiments, the density of the fluid of the reservoir chamber may correspond to a density of phosphate buffered saline, or plasma, or an amount of therapeutic fluid remaining in the reservoir from a prior exchange, or combinations thereof, for example. As described elsewhere herein, differences in fluid density as a result of temperature differences between the exchanged fluids can improve bottom-up filling efficiency. As mentioned above, implant orientation and/or tilt angle between the implant and the exchange needle during refilling can improve refill efficiency where there is a solution density different between the fluid being injected and the contents of the implant. Aspiration can be incorporated to aid in the efficiency of the exchange as well.

When injected into a device 100 implanted within the patient, the distance 204 may correspond to no more than approximately the length of the device 100. The distance 204 may be substantially the length of the reservoir 160 so as to place the distal tip 212 near, but not touching the porous structure 120, and the needle structure 201 of the exchange device 200 can be aligned with an elongate axis 100A of the implantable device 100. In many embodiments, the distance 204 may correspond to no more than about half the distance of the reservoir 160 such that the needle structure 201 can be readily aligned with the implantable device. Work in relation to embodiments suggests that a distance providing a tolerance for angular alignment error of the axis 100A with the axis 202 can facilitate exchange and improve efficiency of the exchange. The distance 204 from stop 240 to tip 212 can be no more than about half of the axial distance of the implantable device can facilitate alignment during injection.

The intermediate portion 220 can include an extension 222 extending between the tapered portion 224 and the distal portion 210. The extension 222 can have a cross-sectional size that is smaller than the tapered portion 224. The extension 222 can have a smooth outer surface to penetrate tissue. The tapered portion 224 can have a smooth outer surface to penetrate tissue and the septum 140 of the device 100. The outer surface of the tapered portion 224 can extend at an angle of inclination relative to the axis 202, and the tapered portion 224 can include a conic section having an angle with the axis such that the outer surface extends at the angle of inclination relative the axis. The angle of inclination of the tapered portion 224 can be no more than about 25 degrees, for example. The angle of inclination can be about 1 degree, about 2 degrees, about 5 degrees, about 10 degrees, about 15 degrees, about 20 degrees, or about 25 degrees, for example. The extension portion 216 (i.e., the distal end region of the inner cannula 270 extending distally past the distal end of the outer cannula 280) can have a first cross-sectional dimension, and the portion of the outer cannula 180 having the plurality of side ports 236 can have a second cross sectional dimension greater than the first dimension, such that tapered portion 224 having the angle of inclination extends therebetween to connect the extension portion 216 with the portion having the plurality of side ports 236.

Still with respect to FIG. 6B, the proximal portion 230 can include the plurality of openings or side ports 236 spaced apart along the axis 202 and distributed circumferentially around a longitudinal axis of the proximal portion of the outer cannula 280 to receive fluid from a plurality of circumferential and axial locations when the stop 240 engages the conjunctiva 16 to place the plurality of openings within the reservoir chamber. At least one side port 237 of the plurality of side ports 236 can be separated from the stop 240 with a distance 206 corresponding substantially to the thickness of the septum 140, such that the at least one side port 237 of the plurality of side ports 236 can be placed near the inner surface of the septum 140 to receive fluid contacting the inner surface of the septum 140. In some implementations, the height of the septum 140 from the lower surface 142 to the upper surface 144 is within a range from about 0.25 to about 2 mm, about 0.5 to about 1.5 mm, or about 1.0 mm to about 1.3 mm, preferably about 1.1 mm to about 1.2 mm, such that the thickness of the septum 140 is substantially greater than a thickness of the conjunctiva which can be approximately 100 μm. The distance 206 corresponding substantially to the thickness of the septum 140 can correspond substantially to the thickness of the septum 140 and the epithelium of the patient.

As mentioned, the outer cannula 280 can be configured to extend over at least a portion of the inner cannula 270. The outer cannula 280 can extend along the intermediate portion 220 and the proximal portion 230, and the inner cannula 270 can extend through the outer cannula 280. The outer cannula 280 can include the plurality of side ports 236 and provide one or more channels extending along inner cannula 270 to pass the fluid of the implantable device through the septum 140.

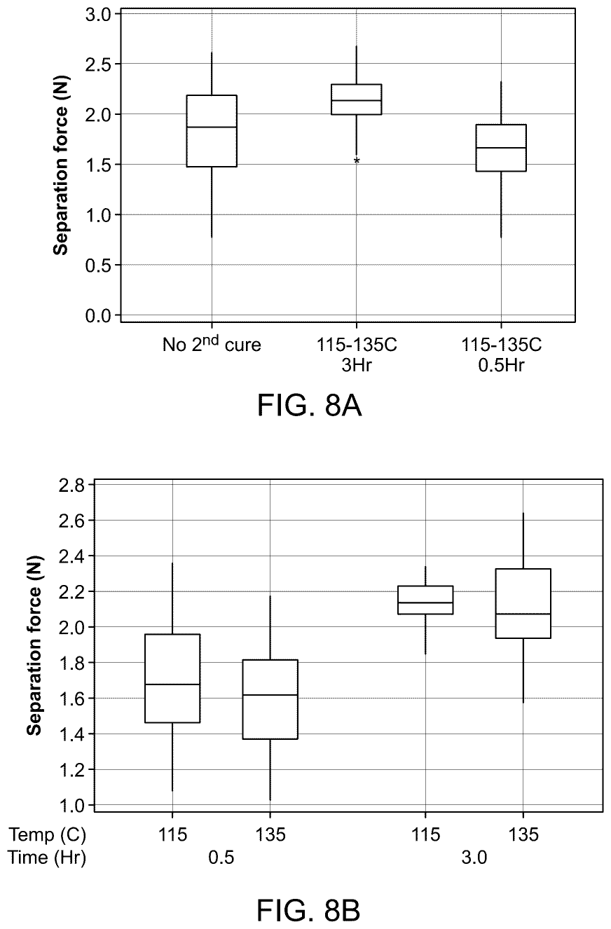

FIG. 6C illustrates a cross-sectional view of a needle structure 201 of the exchange needle device 200 having the outer cannula 280 extending over the inner cannula 270. The inner cannula 270 can include a channel 219, for example a bore or a lumen, extending distally to the distal opening 214 at a distal end region of the inner cannula 270 and proximal to a connector to couple the channel 219 to a syringe holding the therapeutic fluid to be injected into the device 100. The outer cannula 280 can include portions corresponding to the intermediate and proximal portions of the needle structure 201. The extension 222 of the outer cannula 280 can include a distal portion of the outer cannula 280 having an inner surface sized to engage an outer surface of the inner cannula 270. In some implementations, the diameter of extension 222 can have an inner diameter that approaches an outer diameter of the inner cannula 270 to engage the inner cannula 270 with at least one of pressure or friction such that substantially no annular space exists between them at the distal-most end of the outer cannula 280. This minimizes distal-facing space between the inner cannula 270 and the outer cannula 280 that can contribute to coring of the septum 140 upon insertion of the needle structure 201 into the device 100. The tapered portion 224 can include an intermediate portion of outer cannula 280, in which the outer cannula 280 has a tapered surface to penetrate the tissue and septum 140. The proximal portion 230 can include a proximal portion of the outer cannula 280 having the plurality of side ports 236 and the extension 238. As best shown in FIG. 8C, an annular channel 239 can extend along an outer surface of the inner cannula 270 to the plurality of side ports 236. The channel 239 can extend proximally along extension portion 238 toward a collection chamber 250 to receive the fluid of the implantable device 100. The annular space or channel 239 extending between the cannulae 270, 280 is located a distance proximal to the distal-most end of the outer cannula 280 and begins near the location of the tapered section 224 and the flaring outward of the outer cannula 280 away from the inner cannula 270 near the proximal portion 230 and the location of the side ports 236. The tight tolerance between the inner diameter of the outer cannula 280 at the location of the extension 222 and the outer diameter of the inner cannula 270 at the location of the extension 222 reduces coring and damage of the septum 140 upon insertion of the needle structure 201 into the device. The channel 239 can couple the plurality of side ports 236 to the collection chamber 250 to receive the fluid of the implantable device 100.

It should be appreciated that the tapered portion 224 can change in outer diameter according to a variety of geometries and use of the term “taper” or “tapered” is not intended to be limiting to any particular shape. The tapered section 224 can have a greater outer diameter at a proximal end region of the tapered section 224 compared to a smaller outer diameter at a distal end region of the tapered section 224. The change in outer diameter can, but need not be constant. For example, the tapered section 224 can incorporate one or more cylindrical regions between the proximal-most end of the tapered section 224 and the distal-most end of the tapered section 224. The tapered section 224 can be a linear taper or conical in shape or can be curved or have the shape of an arc or ogive.

As mentioned, the exchange device 200 can include or be configured to couple to a syringe or other container configured to hold fluid to be delivered to the reservoir 160. FIG. 6D shows an implementation of an exchange device 200 having a proximal hub or connector 290 configured to couple to a syringe. The connector 290 can be a locking connector having an extension 292 sized to fit in a channel of connector of syringe, for example. The exchange device 200 can include components of a standard locking needle assembly, for example a standard locking needle such as a Luer-Lok™ fitting or a pressure fit connector. Alternatively, the connector 290 may include a non-standard connector to limit access to the exchange device 200. For example, the connector 290 can be a star connector or other connector, and connector 290 may include a lock and key mechanism. The lock and key mechanism can have a lock on the exchange device 200 configured to receive a key of the injector, such that the lock of connector 290 can receive the key of connector to couple the injector to the exchange device 200 and permit injection from chamber through opening 214. Alternatively, the syringe may be affixed to exchange device 200, and syringe provided with a single dose of therapeutic agent.

The exchange device 200 also includes a collection chamber 250 configured to receive fluid from the reservoir 160. The collection chamber 250 can be defined by a wall 252 configured to surround the inner cannula 270 extending through the outer cannula 280. The wall 252 can extend a substantial distance from the stop 240 and can include at least one opening 258 that can vent to atmospheric pressure.

An outlet channel 254 can extend from container 250 to the at least one vent opening 258 to atmospheric pressure.

Again with respect to FIGS. 6A-6D, the outer cannula 280 can be configured in many ways and can have a wall thickness from about 0.250 microns to about 250 microns, for example about 25 microns (0.001 inches or 1/1000 inch). The outer cannula 280 can include an inside diameter sized larger than the outside diameter of inner cannula 270 so as to provide an annular channel 239 extending axially between the inner cannula 270 and the outer cannula 280 from the plurality of side ports 236 to the opening 285. The inner cannula 270 can be as large as about 20 gauge (maximum OD of about 0.92 mm) depending on the size of the septum being penetrated. A smaller size is preferred for a smaller septum, for example, 30 gauge or 34 gauge received within suitably larger outer cannula 280 (e.g., 28 gauge). The size of the inner and outer cannulae are limited by the diameter of the septum 140 being penetrated. The elongate needle structure 201 formed by the inner and outer cannulae as a maximum outer diameter that is preferably no greater than about 50% the size of the septum diameter. For example, a septum of about 1.1 mm in diameter can receive an elongate needle structure 201 that is as large as about 25 gauge (maximum outer diameter of 0.53 mm), although smaller sizes are preferred. The diameter of each of the side ports 236 can be within a range from about 2.5 mm down to about 3 microns, for example within a range from about 25 microns to about 250 microns, or about 50 microns to about 150 microns. The diameter of each of the plurality of side ports 236 can be uniform or can vary in size as well as shape.

The plurality of side ports 236 can be one or more of many shapes and can be arranged in many ways. Each column can include from about 1 to about 20 holes, for example, and can be circular, oval, elliptical or other shapes, for example. The outer cannula 280 can include four columns of circular holes forming the side ports 236. Each of the side ports 236 can have a diameter of no more than about one half of the thickness of the outside diameter of the outer cannula 280, for example, and may be located circumferentially at 90 degrees to each other, for example. Each of the four columns may extend axially along the outer cannula 280. The columns can be spaced angularly at 90 degrees to each other, for example. The outer cannula 280 can include two columns, each column comprising about four side ports 236, each side port 236 having a diameter of no more than about one eighth of the diameter of the outside diameter of the outer cannula 280. The two columns may be spaced apart circumferentially at 180 degrees, and the side ports 236 can include holes cross-drilled through both sides of the outer cannula 280, such that each hole has a corresponding hole on the other column on an opposite side of the sheath. The outer cannula 280 can include about four cross-drilled holes, each hole having a diameter of no more than about three quarters of the diameter of the outside diameter of the outer cannula 280, for example. The holes can be pairs of holes, in which the holes of each pair have corresponding axial locations. The holes can be arranged in two columns spaced circumferentially at 180 degrees. The outer cannula 280 can include at least about three columns of at least about 3 holes, each hole having a diameter of no more than about one quarter of the diameter of the outside diameter of the outer cannula 280. The columns can be spaced apart circumferentially at about 120 degrees, for example. The outer cannula 280 can include at least about 40 side ports 236, each side port 236 having a diameter of no more than about one tenth of the diameter of the outside diameter of the outer cannula 280.

The arrangement of the opening 214 from the inner cannula 270 can vary as well. For example, the opening 214 can be configured to change direction of flow from the inner cannula 270 into the reservoir 160 to impact refill efficiency. The opening 214 can include one or more side openings located near the distal tip 212 of the inner cannula 270. The side ports 236 in the outer cannula 280, the openings 214 in the inner cannula 270, the density/viscosity of the therapeutic fluid being injected, the presence of one or more flow director type features within the device 100 can all impact the effective flow patterns within the device to improve exchange efficiency.

Again with respect to FIG. 6D, the collection chamber 250 of the exchange needle device 200 can have a volume (e.g., no more than about 200 uL, or no more than about 150 uL, or no more than about 100 uL, or no more than about 50 uL). A porous structure can be located within at least a region of the collection chamber 250 along the vent path or the vent opening 258 can be open without any porous structure. The porous structure can be formed of a material having a low resistance to air and other gasses while substantially inhibiting flow of a liquid, such as the liquid from the device 100.

The exchange needle apparatus can impact the integrity of the septum 140 upon repeated penetrations. For example, if there is an annular space between the inner cannula 270 and outer cannula 280 at a distal end region of the needle structure, penetration of the septum 140 by the exchange apparatus can contributed to coring of the septum 140. The pressure and/or friction of the exchange needle apparatus penetrating the septum 140 can also impact the device 100 for long-term retention and treatment.

As mentioned above, the needle structure 201 of the exchange needle device 200 can incorporate a lubricious coating 203 along at least a portion of its working length to reduce friction during penetration of the septum 140. The lubricious coating can be on the outer surface of the outer cannula 280 and the outer surface of the inner cannula 270 extending distal to the distal end of the outer cannula 280.

The coating 203 can be a silicone material, including silicone oils and partially cross-linked silicone materials with plasma. Other lubricants may be used including polydimethylsiloxane (PDMS) or perfluoropolyethers. The coating 203 coats the external surfaces of the needle structure 201 while maintaining the patency of the side ports 236 and distal opening 214. The length can vary from about 3.5 mm up to about 4.5 mm from the distal-most tip 212 of the inner cannula 270 up to a region near the stop 240. FIG. 6E illustrates the needle 210 including the outer cannula 280 surrounding a proximal end region of the inner cannula 270 and the distal tip 212. The total length of the needle 210 from the stop 240 to the distal tip 212 can be about 4.0 mm to about 5.0 mm. The outer cannula 280 can have a length that is about 4 mm from the stop 240 to its distal-most end. The inner cannula 270 can extend a distance past the distal-most end of the outer cannula 280 so that its distal tip 212 is available for penetration of the septum 140. The coating 203 can extend from the distal tip 212 up to a region just short of the location where the needle structure 201 extends outside the stop 240, or about 4.0 mm to about 4.25 mm. The coating 203 of the needle structure 201 in combination with improved bonding of the septum 140 to the bore 180 of the body 130 and improved bonding of the septum 140 to the encasement 110 all contribute to improved septum retention with repeated penetrations.

### EXPERIMENTAL

### Example 1

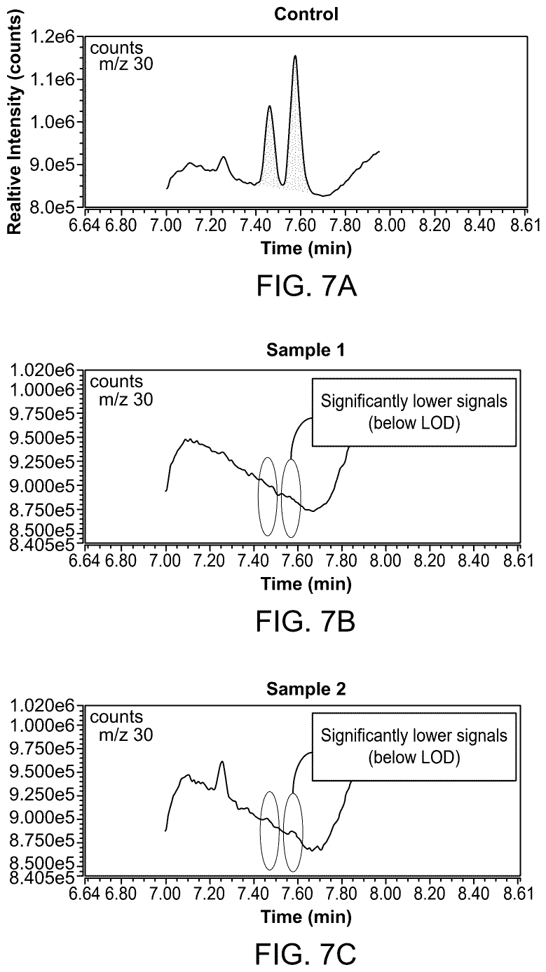

The effectiveness of the second stage of curing on improved septum bonding was demonstrated by measuring residual TMHMD in subassemblies using gas chromatography/mass spectroscopy (GCMS). All subassemblies (i.e., proximal body 130 with bonded septum 140 and no overmolded encasement 110) underwent a first stage of curing of the epoxy (Epotek) dispensed to adhere the silicone septum within the bore of the polysulfone proximal body. The first stage of curing included oven heating bonded subassemblies at 67° C. for 3 hours. A first group of subassemblies underwent no second stage of curing (see control of FIG. 7A). A second group of subassemblies underwent a second stage of curing by oven heating at 150° C. for 3 hours (see Sample 1 of FIG. 7B). A third group of subassemblies underwent a second stage of curing by oven heating at 125° C. for 3 hours (see Sample 2 of FIG. 7C). Following curing, five subassemblies of each group were pooled in 0.5 mL methanol in glass vials and agitated at 300 rpm for 30 minutes at ambient temperature. The extracts were tested using GCMS to assess residual 2,2,4(2,4,4)-Trimethyl-1,6-hexanediamine (TMHMD) (CAS-No 25513-64-8 at 60-100% Epoxy part B). The subassemblies of the second and third groups that went through both the first stage of curing and the second stage of curing showed significantly lower signals below LOD (limit of detection or the lowest concentration of the analyte in the test sample that is easily distinguished from zero) for TMHMD residuals within the proximal body compared to control subassemblies (i.e., FIG. 7A) in which only a first stage of curing was performed.

### Example 2

The force necessary to separate an overmolded silicone encasement bonded to an upper surface of a silicone septum epoxied within a bore of a polysulfone body was measured to assess the impact of the second stage of curing on bond strength between the septum and the encasement. Septum/body subassemblies were constructed using only a single stage of curing or both a first stage and a second stage of curing. The first stage of curing included oven heating subassemblies at 67° C. for 3 hours. The second stage of curing included oven heating subassemblies at 115° C. for 0.5 hour, at 115° C. for 3 hours, or 135° C. for 0.5 hour, or 135° C. for 3 hours. Following the cure protocol (whether just one stage of curing or two stages of curing), the elastomeric silicone encasement was overmolded and bonded to the upper surface of the septum. An external region of the encasement encapsulating the flange of the proximal body was cut so that the encasement could be inverted and disengaged from the flange to evaluate the bond between the upper surface of the septum and the lower surface of the encasement alone. The proximal body of the subassembly was held within an implant holding fixture and the inverted encasement clamped and pulled upward away from the upper surface of the septum thereby tensioning the bond between the encasement and the septum. The maximum force required to separate the encasement from the septum was recorded.

FIG. 8A shows separation force in Newtons (N) for a subassembly without a second stage of curing (see, No 2^{nd} cure) compared to two groups of subassemblies having undergone both a first stage of curing and a second stage of curing. The subassemblies in the first group were heated to 115° C. or 135° C. and results pooled in FIG. 8A. Similarly, the subassemblies in the second group were heated to 115° C. or 135° C. and results pooled in FIG. 8A. The second epoxy cure at the longer period of 3 hours significantly increased the force necessary to separate the overmolded encasement from the upper surface of the septum. The second epoxy cure at the shorter period of time (i.e., 0.5 hour) did not significantly alter the separation force needed to pull the overmolded encasement from the upper surface of the septum.

FIG. 8B shows the effect of temperature and time of the second stage of curing on the separation force in Newtons (N). The second stage of epoxy curing compared 115° C. and 135° C. at 0.5 hour and 3 hours. The subassemblies with a second stage of epoxy curing at the shorter time-period had lower separation force needed to break the bond between the encasement and the septum compared to subassemblies with a second stage of epoxy curing at the longer time-period, even at the higher temperature curing. The length of the second stage of curing rather than the higher temperature increased the robustness of the bond between the overmold encasement and the upper surface of the septum.

### Example 3

The performance testing protocol included as a first step puncturing each implant of each group with a refill needle apparatus and as a second step incubating in phosphate buffered saline (PBS) at an elevated temperature (e.g., 80° C.). As a third step, each implant underwent another puncture at a randomly selected location across the upper surface of the septum. The second and third steps were repeated every 3.5 days until the sample leaks under pressure. Low pressure air was applied with a test apparatus at approximately 0.3 psi to an interior of the implant and leaks assessed by an operator using a microscope to visually inspect the devices submerged in a liquid for continuous formation of air bubbles (e.g., bubbles continue to grow in size over 60 s). Another failure mode includes visual inspection with the unaided eye for missing or dislodged septum. Performance of the implants are described below.

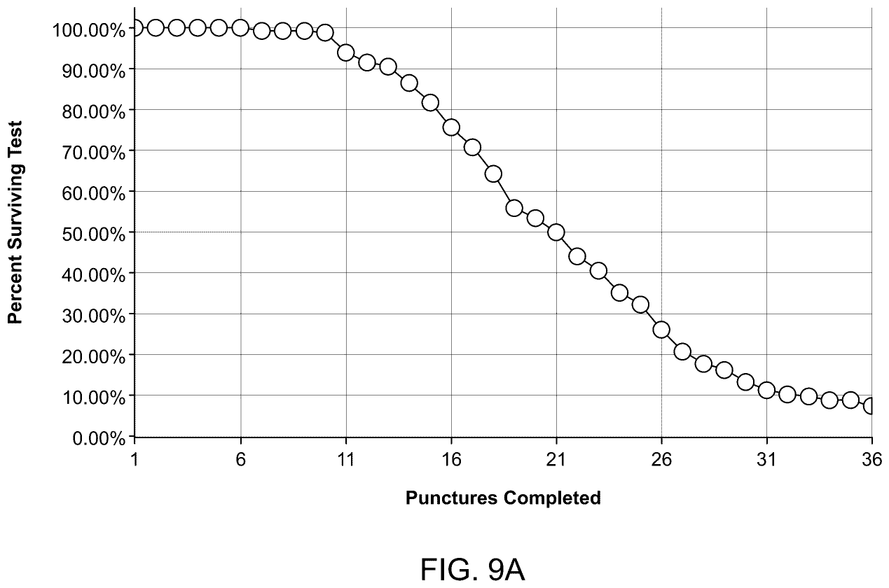

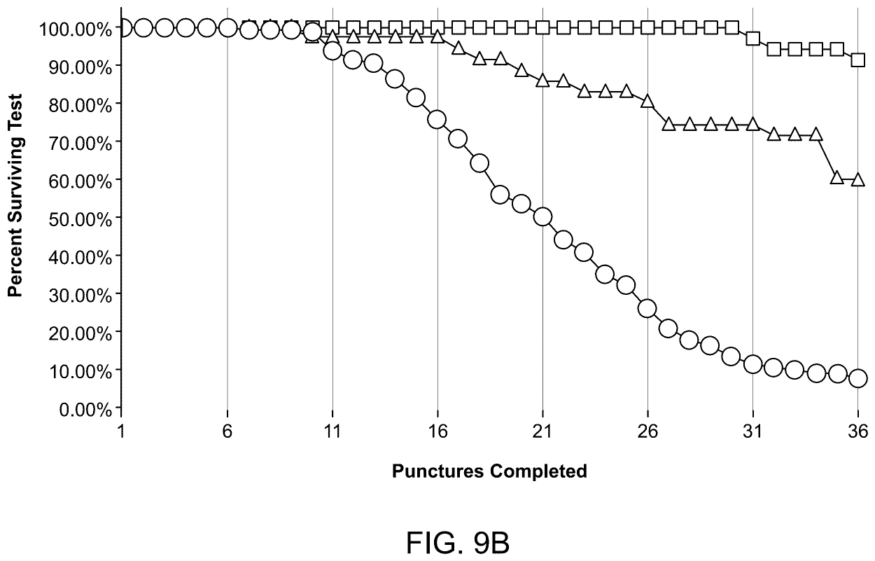

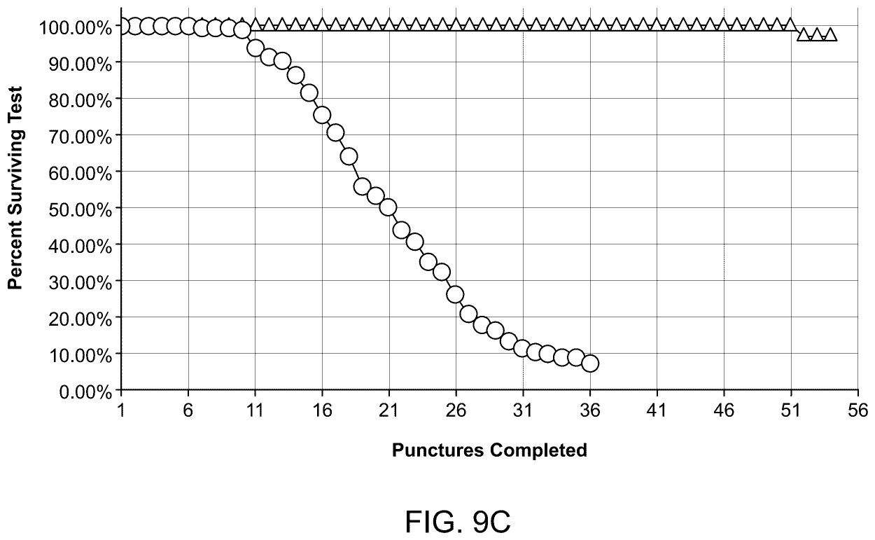

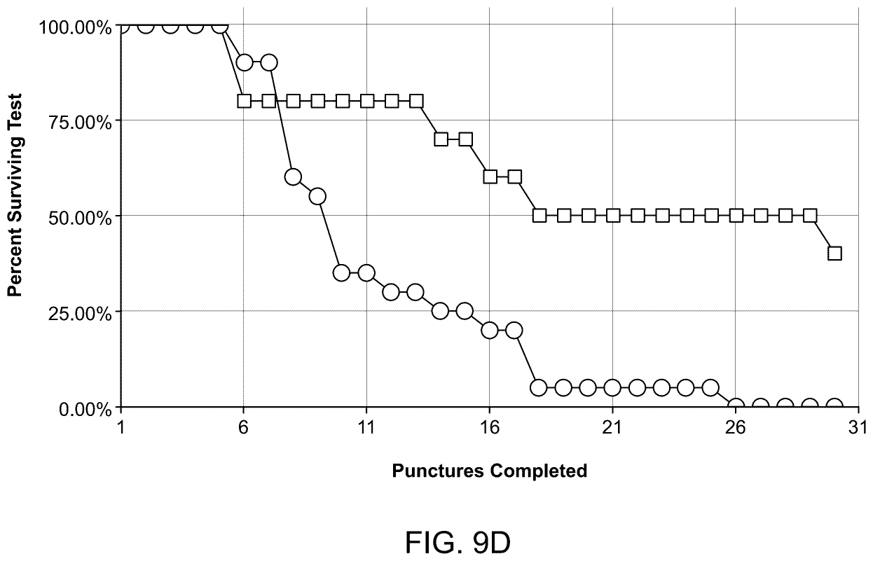

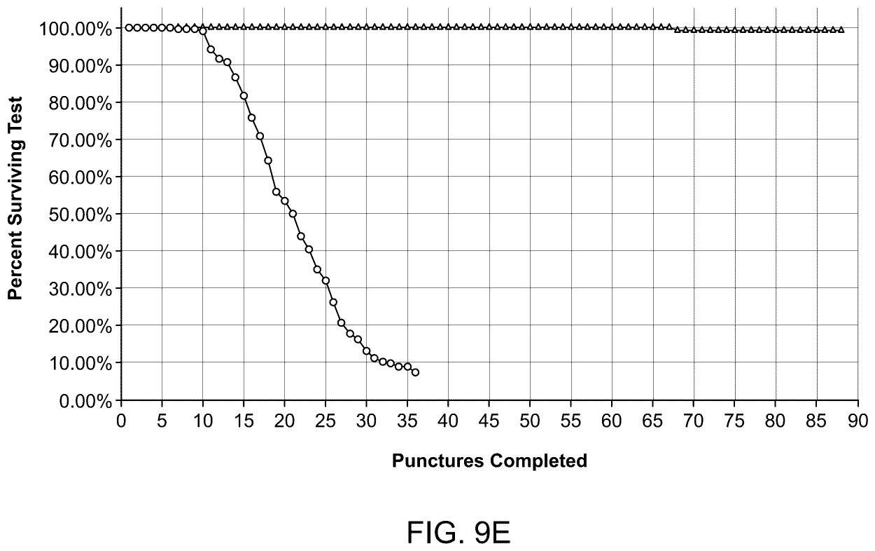

FIG. 9A shows performance of implants having a septum repeatedly penetrated by an uncoated refill needle. The septum implants had an upper surface trimmed only along the central region such that the outer perimeter region remained untrimmed. Further, the untrimmed outer perimeter region of the shorter septum implants lay below the plane of the upper surface of the flange. The trim height of the septum from the lower surface to the planar upper surface at the central region was about 1.14 mm. Approximately 50% of the implants tested (n=188) survived to 21 punctures with the uncoated refill needle. At 36 punctures, which is equivalent of more than about 16 years of implantation, less than 10% survived.

FIG. 9B shows performance of implants having the same septum as in FIG. 9A (circles) penetrated by an uncoated refill needle compared to implants penetrated by a first refill needle that had a low level of siliconization to reduce insertion force by 25% (triangles) and a second refill needle that had a medium level of siliconization to reduce insertion force by 50% (squares). Low level of siliconization was provided by 0.5% solution of 1,000 cS silicone fluid and medium level of siliconization was provided by 5.0% solution of 12,500 cS silicone fluid.

Reducing refill needle insertion force by 25% improved the survival of the implant from 10% as in FIG. 9A to 60% after 36 punctures (n=35). Reducing refill needle insertion force by 50% resulted in a 91% survival through 36 punctures (n=35).

FIG. 9C shows performance of implants having a longer trim height septum and punctured with silicone refill needle (triangles) compared to the shorter trim height septum punctured with non-siliconized refill needle (circles) as in FIG. 9A. The siliconized refill needles had 50% less insertion force compared to the unsiliconized refill needles. The implants with the longer septum had double the internal septum bond strength compared to the shorter septum implants. Implant survival at 54 punctures improved from 10% to 97% (n=35).

FIG. 9D shows performance of implants having an upper surface trimmed only along the central region such that the outer perimeter region remained untrimmed and lay below the plane of the upper surface of the flange. The septum trim height of the implants was about 1.05 mm. The survival of implants after puncture with non-siliconized refill needle (circles) and siliconized refill needle (squares) was compared. Implant survival after 18 punctures in the siliconized refill needle group plateaued at about 50% compared to the unsiliconized refill needle group, which had substantially failed by 18 punctures. The siliconized refill need group had about 37% survival after 30 punctures.

FIG. 9E shows performance of implants as in FIG. 9A punctured with nonsiliconized refill needle (circles) compared to improved implants having increased septum contact area, low epoxy, and extra oven curing punctured with siliconized refill needle (triangles). After 36 punctures, which is the equivalent of more than about 16 years of use, the implants in the siliconized refill needle group maintained about 100% survival. The first failure on puncture occurred after 68 punctures. The septum did not dislodge, but appeared to leak through a hole in the septum.

### Example 4

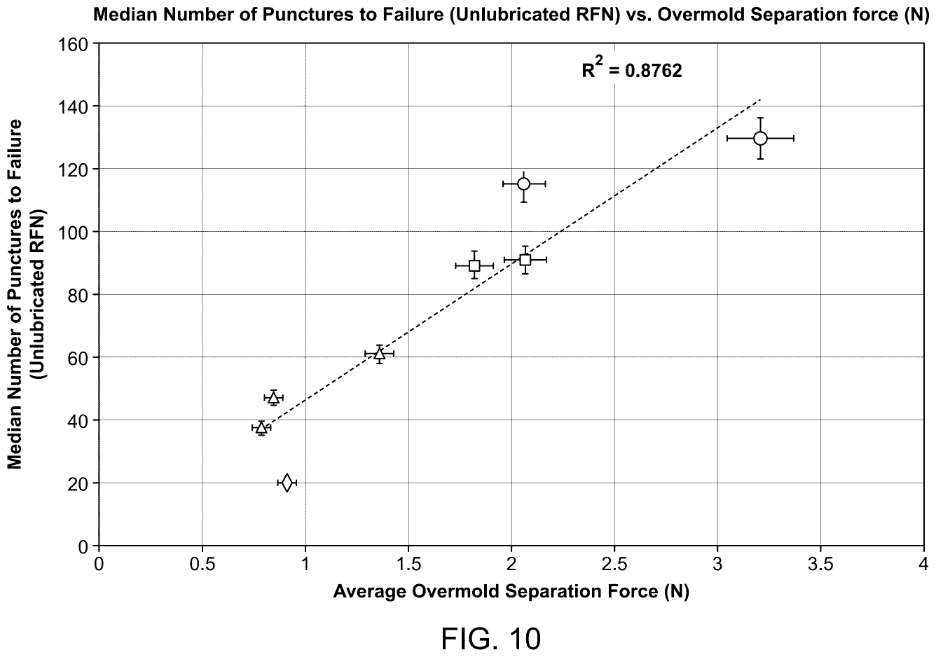

FIG. 10 illustrates the correlation between overmold separation force and punctures-to-failure. The performance testing protocol to assess punctures-to-failure is described above in Example 3 Overmold separation force was measured as described above in Example 2. Overmold separation force in Newtons (N) is shown on the x-axis and punctures-to-failure is shown in the y-axis. Implants having a septum positioned within the bore without using epoxy are shown as filled circles. Implants having an epoxy bonded septum with a fully trimmed upper surface and outer perimeter substantially flush with the upper surface of the flange are shown as filled squares. Implants having an epoxy bonded septum that is shorter and with a partially trimmed upper surface along the central region and an untrimmed outer perimeter countersunk below the upper surface of the flange are shown as filled triangles. Implants having the shortest epoxy bonded septum with a partially trimmed upper surface are shown as filled diamonds. Implants with a higher overmold separation force have higher punctures-to-failure performance (correlation R2=0.8762). Implants having longer septum heights (squares) bonded better than implants with shortest septum heights (triangles and diamonds). Implants without any epoxy bonding the septum to the proximal body had the best outcome.

The devices described herein can be used to deliver essentially any substance. As used herein, “substance,” “drug” or “therapeutic” is an agent or agents that ameliorate the symptoms of a disease or disorder or ameliorate the disease or disorder including, for example, small molecule drugs, proteins, nucleic acids, polysaccharides, and biologics or combination thereof. Therapeutic agent, therapeutic compound, therapeutic regimen, or chemotherapeutic include conventional drugs and drug therapies, including vaccines, which are known to those skilled in the art.

Therapeutic agents include, but are not limited to, moieties that inhibit cell growth or promote cell death, that can be activated to inhibit cell growth or promote cell death, or that activate another agent to inhibit cell growth or promote cell death. Optionally, the therapeutic agent can exhibit or manifest additional properties, such as, properties that permit its use as an imaging agent, as described elsewhere herein.

In aspects, description is made with reference to the figures. However, certain aspects may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detain in order to not unnecessarily obscure the description. Reference throughout this specification to “one embodiment,” “an embodiment,” “an aspect,” “one aspect,” “one implementation, ”an implementation,“ or the like, means that a particular feature, structure, configuration, or characteristic described is included in at least one embodiment, aspect, or implementation. Thus, the appearance of the phrase ”one embodiment,“ ”an embodiment,“ ”one aspect,“ ”an aspect,“ ”one implementation, “an implementation,” or the like, in various placed throughout this specification are not necessarily referring to the same embodiment, aspect, or implementation. Furthermore, the particular features, structures, configurations, or characteristics may be combined in any suitable manner in one or more implementations.

The use of relative terms throughout the description may denote a relative position or direction or orientation and is not intended to be limiting. For example, “distal” may indicate a first direction away from a reference point. Similarly, “proximal” may indicate a location in a second direction opposite to the first direction. Use of the terms “front,” “side,” and “back” as well as “anterior,” “posterior,” “caudal,” “cephalad” and the like or used to establish relative frames of reference, and are not intended to limit the use or orientation of any of the devices described herein in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples, embodiments, aspects, and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

1. An ophthalmic drug delivery device comprising:
a body defining a refillable reservoir;
an extrascleral flange projecting from a proximal end of the body and defining a bore extending from an upper surface of the flange into the reservoir defined by the body;
a septum comprising an upper surface connected to a lower surface by a curved outer surface, wherein the curved outer surface of the septum forms a first bond with the bore, wherein a perimeter region of the upper surface of the septum lies flush with a plane of the upper surface of the flange and a central region of the upper surface lies below the perimeter region; and
an elastomeric encasement extending over at least the upper surface of the flange and the upper surface of the septum, wherein the encasement prevents displacement of the septum relative to the bore upon penetration by a needle.

2.-3. (canceled)

4. The device of claim 1, wherein a maximum height of the septum between the lower surface and the upper surface is greater than 1.05 mm and less than 1.35 mm.

5. The device of claim 1, wherein the central region of the upper surface of the septum forms a depression in the upper surface of the septum, wherein the encasement fills the depression.

6. The device of claim 1, wherein the encasement and the upper surface of the septum interface along a full diameter of the septum.

7. The device of claim 6, wherein a contact area at the interface is non-planar.

8. The device of claim 1, wherein the septum is formed of silicone elastomer.

9. The device of claim 1, wherein the body is formed of polysulfone.

10. The device of claim 1, wherein the encasement is formed of silicone elastomer.

11. The device of claim 1, wherein the encasement bonds only to the upper surface of the septum forming a second bond.

12. The device of claim 11, wherein the first bond between the curved outer surface of the septum and the bore is formed using an epoxy adhesive.

13. The device of claim 12, wherein the epoxy adhesive comprises an amine-based epoxy curing agent.

14. The device of claim 11, wherein the first bond is substantially free of trimethyl-1,6-Hexandiamine prior to forming the second bond between the encasement and the upper surface of the septum.

15.-17. (canceled)

18. The device of claim 1, wherein the body is transparent or translucent.

19. The device of claim 1, wherein the encasement is transparent or translucent.

20. The device of claim 1, wherein the encasement and the septum are configured to be penetrated by the needle during refilling of the reservoir and configured to reseal after penetration and upon removal of the needle from the reservoir.

21. The device of claim 1, wherein the needle is siliconized along at least a portion of its length.

22. The device of claim 1, wherein the septum is oversized relative to the bore such that the bore applies radial compression on the septum.

23. (canceled)

24. The device of claim 1, wherein the body defining the refillable reservoir is configured for implantation within a vitreous of an eye through a penetration in the sclera of the eye.

25. The device of claim 1, wherein the body further comprises a porous structure positioned within a distal end region of the refillable reservoir.

26. (canceled)

27. The device of claim 1, wherein the encasement encapsulates the upper surface and a lower surface of the flange without bonding to the flange.

28. The device of claim 1, wherein the flange further comprises one or more through-holes.
